(19) United States
(12) Patent Application Publication
(10) Pub. No.: US 2022/0117879 A1
(43) Pub. Date: Apr. 21, 2022

(54) PHOTO-PROTECTIVE PLANT-DERIVED COMPOSITION

(71) Applicants: Ariel Scientific Innovations Ltd., Ariel (IL); The Dead-Sea & Arava Science Center, Doar-Na Yam HaMelach (IL)

(72) Inventors: Shoshana BEN-VALID, Jerusalem (IL); Guy COHEN, Ein Gedi (IL)

(73) Assignees: Ariel Scientific Innovations Ltd., Ariel (IL); The Dead-Sea & Arava Science Center, Doar-Na Yam HaMelach (IL)

(21) Appl. No.: 17/428,685

(22) PCT Filed: Feb. 6, 2020
(86) PCT No.: PCT/IL2020/050150

§ 371 Date: Aug. 5, 2021

Related U.S. Application Data
(60) Provisional application No. 62/802,219, filed on Feb. 7, 2019.

(51) Int. Cl. A61K 8/9789; A61K 8/60; A61Q 17/04

(52) U.S. Cl. CPC A61K 8/9789; A61Q 17/04; A61K 8/60

(57) ABSTRACT

Described herein is a process for obtaining a UV-absorbing material, optionally 1,2,3,4,6-pentagalloyl glucose, from sumac leaves and/or budding fruits, as well as a UV-absorbing material obtained according to said process, and compositions comprising such a UV-absorbing material. The process comprises contacting sumac leaves and/or budding fruits with a water-miscible organic solvent, and removing the water-miscible organic solvent. Compositions described herein sunscreen compositions wherein a concentration of the UV-absorbing material is at least 0.005 mg/ml, as well as pharmaceutical and/or cosmetic compositions.

### RELATED APPLICATION

This application claims the benefit of priority of U.S. Provisional Application No. 62/802,219 filed on Feb. 7, 2019, the contents of which are incorporated by reference as if fully set forth herein.

### FIELD AND BACKGROUND OF THE INVENTION

The present invention, in some embodiments thereof, relates to photo-protection, and more particularly, but not exclusively, to UV-blocking composition which can be obtained from plant material.

Chronic exposure to solar radiation, and in particular to its ultraviolet B (UVB) region, directly affecting the epidermal surfaces and inner layers, is linked to several pathophysiological alterations, including sunburns, erythema, modulation of the immune response and increases exogenous skin aging [Matsurama & Anathaswamy, Toxicol Appl Pharmacol 2004, 195:298-308; Kraemer, Proc Natl Acad Sci USA 1997, 94:11-14; Lavker et al., J Am Acad Dermatol 1995, 32:53-62; Portugal-Cohen et al., Exp Dermatol 2009, 18:781-789]. Importantly, UVB may also harm the skin by inducing cellular apoptosis and damage the DNA of the epidermal cells, which is considered one of the major risk factor for the development of skin cancer [Tuorkey, Eur J Cancer Prev 2015, 24:430-438].

Sunscreens are used in defense against solar radiation, not only as an important tool against skin cancer, but also to alleviate other skin ailments induced by solar radiation, such as aging, wrinkle formation, undesired pigmentation and collagen loss [Mancebo et al., Dermatol Clin 2014, 32:427-428]. However, it has been reported that currently used commercial sunscreens can penetrate and permeate the skin, reaching the circulatory system and producing systemic damage to the body [Touitou & Godin, Clin Dermatol 2008, 26:375-379]. Skin that is treated with sunscreens and subsequently exposed to UV radiation paradoxically exhibits an exacerbated presence of ROS (reactive oxygen species) [Hanson et al., Free Radic Biol Med 2006, 41:1205-1212]. This points both to sunscreens' ability to penetrate the outermost skin layers, and to the pro-radical effect of some sunscreens. In addition, sunscreens and their degradation byproducts had been detected in urine and breast milk of the individuals who apply cosmetics with sunscreens [Hayden et al., Lancet 1997, 350:863-864]. The ability of some sunscreens to bind and activate estrogen or thyroid receptors has also been reported [Gilbert et al., Int J Cosmet Sci 2013, 35:208-219].

Sunscreens exhibit significantly toxicity for human users, but also possess an ecological and existential threat to corals and coral reefs, which are polluted by sunscreens on swimmers or carried in wastewater [Danovaro et al., Environ Health Perspect 2008, 116:441-447].

Sumac (meaning red in Syriac) refers to plants in the genus Rhus and related genera in the Anacardiaciae family.

Rhus coriaria, a particularly well-known species of sumac, is typically a 1-3 meter high shrub or small tree, whose leaves are imparipinnate with 9-15 leaflets, whose inflorescence is a compact and erect panicle, with small and greenish white flowers, and a fruit which is a villose, reddish, 1-seeded drupe. Rhus coriaria grows wild in Mediterranean maquis and forests in any type of soil that is deep and well-drained.

The main use of sumac is as a spice, popular in the Middle East, produced from its sour fruits. Immature fruits and seeds are also eaten. In addition, sumac contains coloring matter and tannins, which are used in dying and tanning fine leather, and can dye protein-based textile materials such as silk and wool [Shabbir, J Animal Plant Sci 2012, 22:505-512]. Dyes of various colors can be prepared from different parts of the plant. In addition, oil extracted from the seeds can be used to make candles. Sumac has been reported to possess hydrolysable tannins (including gallotannins), volatile oil, anthocyanin, gallic acid, and flavonoids such as myricetin, quercetin and kaempferol [Mavlyanov et al., Chem Nat Comp 1997, 33:209; Güvenç & Koyuncu, Turkish J Med Sci 1994, 20:11-13; el-Sissi et al., Planta Med 1972, 21:67-71; Mehrdad et al., J AOAC Int 2009, 92:1035-1043].

Sumac has been reported to possess antibacterial [Adwan et al., Asian Pac J Trop Med 2010, 266-269], antifungal [Hashem & Alamri, Saudi J Biol Sci 2010, 17:167-175], antioxidant and chondroprotective [Panico et al., J Med Plant Res 2009, 3:855-861], DNA protective upon chemical insult [Chakraborty et al., Mut Res 2009, 661:10-17], hypoglycemic [Giancarlo et al., Nat Prod Res 2006, 20:882-896], anti-ischemic and vasorelaxant [Beretta et al., Planta Med 2009, 75:1482-1488], properties, and is non-mutagenic [Al-Bataina et al., J Trace Elem Med Biol 2003, 17:85-90].

U.S. Patent Application Publication No. 2010/0215630 describes a use of an extract of anise, astragalus, cilantro, cinnamon, cloves, dill, fenugreek, feverfew, kudzu, licorice, magnolia, marjoram, oregano, paprika, peppermint, popcorn tree, rosemary, sage, spearmint, skullcap, St. John's wort, sumac, tarragon, thyme and/or valerian for protecting insect microbial agents against ultraviolet radiation, to preserve efficacy of insect pathogens.

The term “tannin” refers to polyphenolic biomolecules capable of forming strong complexes with various molecules, typically via hydroxyl or carboxyl groups in the tannin.

Plant-derived tannins typically belong to one of two major classes: hydrolysable tannins, comprising a core carbohydrate esterified with phenolic groups (derived from gallic acid or ellagic acid); and condensed tannins, formed by condensation polymerization of flavonoids. Hydrolysable tannins comprising gallic acid esters are also referred to as “gallotannins”.

The term “tannic acid” encompasses tannins which are polygalloyl esters of glucose or quinic acid. Commercially available tannic acid is usually extracted from tara (Tara spinosa) pods, gallnuts from Chinese sumac (Rhus chinensis) or Aleppo oak (Quercus infectoria), or tanner's sumac (Rhus coriaria) leaves. The number of galloyl moieties typically ranges from 2 to 12, depending on the source, although the chemical formula of tannic acid is often described by convention as that of decagalloyl glucose.

More than 500 molecules composed of galloyl esters of glucose have been identified in more than 20 plant families, ranging from the very simple 1-monogalloyl-β-glucose (glucogallin), with a molecular weight of 332 Da, to complex polymers with molecular weights of over 4000 Da.

Rothman & Henningsen [J Invest Dermatol 1947, 9:307-313] describes the sunburn-protecting effect of p-aminobenzoic acid, and reports that although tannic acid has been frequently used for such a purpose, it suffers from rapid decomposition upon exposure to UV radiation.

U.S. Pat. No. 4,104,368 describes compositions for providing a conditioning and sun-screening effect, comprising a long-chain quaternary ammonium salt and an acidic moiety providing a sun-screening effect, such as p-aminobenzoic acid and its derivatives, salicylic acid and its derivatives, malonic acid and its derivatives, cinnamic acid and its derivatives, tannic and gallic acids, naphthol sulfonic acids, and anthranilic acid.

Tannic acid is also included in lists of UV filters for sunscreen formulations in U.S. Pat. Nos. 5,169,624, 8,703,753, 9,737,472 and 10,064,797.

U.S. Pat. No. 10,111,821 describes phototherapy by administering electromagnetic radiation at specific wavelengths, by filtering part of the electromagnetic radiation spectrum using topically applied compositions. A wide variety of ultraviolet-absorbing molecules are described therein, including pentagalloyl glucose.

U.S. Pat. No. 7,776,915 describes a topical composition for improving appearance of aged skin, comprising a lipoid acid, a carnitine and a carnosine, and optionally additional agents such as antioxidants, anti-glycation agents, collagen-enhancing agents, mitochondrial resuscitants, thioredoxin, glutathione, NADH, anti-inflammatory agents, depigmenting agents, skin-protective lipids, and sunscreen agents. Pentagalloyl glycose is included therein within a long list of anti-inflammatory agents.

U.S. Pat. No. 4,741,915 describes use of gallotannins such as pentagalloyl glucose as antioxidants in foodstuffs and in cosmetic products such as shaving foam, after-shave, body lotion, make-up removing milk, facial cream, suntan lotion and masks.

Additional background art includes Berardini et al. [Rapid Commun Mass Spectrom 2004, 18:2208-2216]; Chuarienthong et al. [Int J Cosmet Sci 2010, 32:99-106]; Cohen et al. [Negev, Dead Sea and Arava Studies 2015, 7:66-74]; Haddock et al. [J Chem Soc Perkin Trans 1 1982, 0:2535-2545]; Nichols & Katiyar [Arch Dermatol Res 2010, 302:71-83]; Ozer et al. [J Ethnopharmacol 2015, 161:86-91]; Schuch et al. [Free Rad Biol Med 2017, 107:110-124]; Schwack & Rudolph [J Photochem Photobiol B Biol 1995, 28:229-234]; and Wineman et al. [J Herbal Med 2015, 5:199-206].

### SUMMARY OF THE INVENTION

According to an aspect of some embodiments of the invention, there is provided a composition comprising a material extracted from sumac (Rhus spp.) leaves and/or budding fruits, wherein the material absorbs ultraviolet and/or blue light.

According to an aspect of some embodiments of the invention, there is provided a sunscreen composition comprising a UV-absorbing material and a dermatologically acceptable carrier, wherein the UV-absorbing material is extracted from sumac (Rhus spp.) leaves and/or budding fruits, and a concentration of the UV-absorbing material in the composition is at least 0.005 mg/ml.

According to an aspect of some embodiments of the invention, there is provided a sunscreen composition comprising 1,2,3,4,6-pentagalloyl glucose isolated from sumac (Rhus spp.) leaves and/or budding fruits, and a dermatologically acceptable carrier, wherein a concentration of the 1,2,3,4,6-pentagalloyl glucose in the composition is at least 0.005 mg/ml.

According to an aspect of some embodiments of the invention, there is provided a pharmaceutical or cosmetic composition comprising a UV-absorbing material extracted from sumac (Rhus spp.) leaves and/or budding fruits, and a dermatologically acceptable carrier.

According to an aspect of some embodiments of the invention, there is provided a pharmaceutical or cosmetic composition comprising 1,2,3,4,6-pentagalloyl glucose isolated from sumac (Rhus spp.) leaves and/or budding fruits, and a dermatologically acceptable carrier.

According to an aspect of some embodiments of the invention, there is provided a process for obtaining a UV-absorbing material, the process comprising contacting sumac (Rhus spp.) leaves and/or budding fruits with a water-miscible organic solvent, and removing the water-miscible organic solvent, to thereby obtain the UV-absorbing material.

According to an aspect of some embodiments of the invention, there is provided a process for obtaining 1,2,3,4,6-pentagalloyl glucose, the process comprising contacting sumac (Rhus spp.) leaves and/or budding fruits with a water-miscible organic solvent, and removing the water-miscible organic solvent, to thereby obtain 1,2,3,4,6-pentagalloyl glucose.

According to an aspect of some embodiments of the invention, there is provided a UV-absorbing material obtained according to the process described herein, according to any of the respective embodiments.

According to an aspect of some embodiments of the invention, there is provided 1,2,3,4,6-pentagalloyl glucose obtained according to the process described herein, according to any of the respective embodiments.

According to some of any of the embodiments described herein, the sumac comprises Rhus coriaria.

According to some of any of the embodiments described herein relating to a composition, a concentration of the UV-absorbing material in the composition is sufficient to absorb at least 90% of radiation at a wavelength of 300 nm over a path length of 1 mm.

According to some of any of the embodiments described herein, the UV-absorbing material comprises at least one compound having a glucose moiety with a plurality of galloyl substituents.

According to some of any of the embodiments described herein relating to at least one compound having a glucose moiety with a plurality of galloyl substituents, the at least one compound comprises 1,2,3,4,6-pentagalloyl glucose.

According to some of any of the embodiments described herein relating to UV-absorbing material which comprises 1,2,3,4,6-pentagalloyl glucose, a purity of the 1,2,3,4,6-pentagalloyl glucose in the UV-absorbing material is at least 95 weight percents.

According to some of any of the embodiments described herein relating to a pharmaceutical or cosmetic composition, the composition is for use in treating a condition selected from the group consisting of skin aging and a wound.

According to some of any of the embodiments described herein relating to a cosmetic composition, the cosmetic composition is a skin rejuvenation composition and/or a peeling composition.

According to some of any of the embodiments described herein relating to a UV-absorbing material, the UV-absorbing material is obtainable by a process comprising contacting the sumac leaves and/or budding fruits with a water-miscible organic solvent, and removing the water-miscible organic solvent, to thereby obtain the UV-absorbing material.

According to some of any of the embodiments described herein relating to a process, the water-miscible organic solvent comprises a C1-4-alcohol.

According to some of any of the embodiments described herein relating to a process, the water-miscible organic solvent comprises ethanol, acetone and/or glycerin.

According to some of any of the embodiments described herein relating to a process, contacting sumac leaves and/or budding fruits with a water-miscible organic solvent comprises extraction effected using a Soxhlet extractor.

According to some of any of the embodiments described herein relating to a process, contacting sumac leaves and/or budding fruits with a water-miscible organic solvent is effected using a ratio of from 5 to 40 ml of water-miscible organic solvent per gram dry weight of sumac leaves and/or budding fruits.

According to some of any of the embodiments described herein relating to a process, the process further comprises partitioning the UV-absorbing material in a polar solvent and a nonpolar solvent, and collecting the fraction which partitions into the polar solvent.

According to some of any of the embodiments described herein relating to a process comprising partitioning in a polar solvent and a nonpolar solvent, the nonpolar solvent comprises an alkane.

According to some of any of the embodiments described herein relating to a process comprising partitioning in a polar solvent and a nonpolar solvent comprising alkane, the alkane comprises hexane.

According to some of any of the embodiments described herein relating to a process comprising partitioning in a polar solvent and a nonpolar solvent, the polar solvent comprises water.

According to some of any of the embodiments described herein relating to a process comprising partitioning in a polar solvent and a nonpolar solvent, the process further comprises partitioning the fraction which partitions into the polar solvent in water and a water-immiscible polar organic solvent, and collecting the fraction which partitions into the water-immiscible polar organic solvent.

According to some of any of the embodiments described herein relating to a process comprising partitioning in water and a water-immiscible polar organic solvent, the water-immiscible polar organic solvent comprises an ester.

According to some of any of the embodiments described herein relating to a process comprising partitioning in water and a water-immiscible polar organic solvent which comprises an ester, the ester comprises ethyl acetate.

According to some of any of the embodiments described herein relating to a process comprising partitioning in water and a water-immiscible polar organic solvent, the process further comprises effecting crystallization of the UV-absorbing material by contacting the fraction which partitions into the water-immiscible polar organic solvent with a solvent comprising water.

According to some of any of the embodiments described herein relating to a process, the process further comprises purifying the UV-absorbing material by column chromatography. Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a bar graph showing the SPF values of ethanol extracts (0.1 mg/ml) of branches, twigs, leaves, budding fruits, ripe fruits and roots of Rhus coriaria from various geographical locations (in Israel).

FIG. 2 schematically depicts a procedure for extracting an exemplary UV-absorbing substance (SH-101) from Rhus coriaria leaves according to exemplary embodiments of the invention.

FIG. 3 is a graph showing the SPF value and percent yield of material extracted from 50 grams dry Rhus coriaria leaf powder as described in FIG. 2, as a function of ethanol volume (in ml) used to obtain an ethanolic extract.

FIGS. 4A-4C present an HPLC chromatogram (absorption at 290 nm) of an exemplary UV-absorbing substance (SH-401) extracted from Rhus coriaria leaves according to some embodiments of the invention (FIGS. 4A and 4B), and a UV absorption spectrum (FIG. 4C) of an exemplary substance associated with the main peak of the HPLC chromatogram (presented results are representative of 3 experiments).

FIG. 5 depicts a structure of an exemplary UV-absorbing compound (pentagalloyl glucose) extracted from Rhus coriaria leaves, as determined by NMR analysis.

FIG. 6 is a bar graph showing SPF values of an exemplary substance isolated from Rhus coriaria (SH-401), crude Rhus coriaria extract, and commercial UVB filters, at concentrations of 0.01 mg/ml (n=3).

FIG. 7 is a bar graph showing SPF values of SH-401 (at a concentration of 0.01 mg/ml) as a function of time of exposure of a film of SH-101 to UV radiation (n=3).

FIGS. 8A-8C present graphs showing epidermal cell viability (as determined by MTT assay) upon incubation of human skin explants with SH-401, as a function of SH-401 (Compound) concentration (FIG. 8A), and levels of apoptosis (FIG. 8B), and TNFα (FIG. 8C), in human skin explants 24 hours after exposure to UVB radiation (350 mJ/cm²) of cells previously treated with 0.02, 0.1, 0.2, 1 or 2 μg/cm² SH-401 or commercial SPF 30 composition (Dr. Fisher) (* p<0.05 relative to non-irradiated vehicle control (0); # p<0.05 relative to irradiated vehicle control (UV); n=3).

FIG. 9 is a bar graph showing levels of ROS (reactive oxygen species) generation (in arbitrary units), as determined by DCFDA (dichlorofluorescin diacetate) assay, in skin explants treated with 0, 0.02, 0.1, 0.2, 1 or 2 μg/cm² of SH-101, with or without UVB radiation (n=3; * p<0.05 relative to treatment with UVB and 0 μg/cm² of SH-101).

FIG. 10 is a bar graph showing levels of lipid peroxidation, measured as MDA (malondialdehyde) concentration as determined by ELISA assay, in skin explants treated with 0, 0.02, 0.1, 0.2, 1 or 2 μg/cm² of SH-101, with or without UVB radiation (n=3; * p<0.05 relative to treatment with UVB and 0 μg/cm² of SH-101).

FIG. 11 is a bar graph showing the ability of SH-401) to scavenge reactive oxygen species (measured as micromole Trolox equivalent units) at concentrations of 0.1, 0.5 or 1 weight percent, as determined based on color shift of DPPH (diphenylpicrylhydrazyl) (vehicle with 0% SH-401 served as a control; n=3; * p<0.05 relative to vehicle control).

FIG. 12 presents images of epidermis cells subjected to a COMET DNA fragmentation assay, following treatment of skin explants with UVB irradiation (350 mJ/cm²) and/or incubation with 2 μg/cm² of SH-401 (Compound) (after treatment of skin, epidermis was peeled and single cell preparations obtained with EDTA; images are representative of 3 experiments; arrows indicate “tail” of damaged cells).

FIG. 13 is a bar graph showing levels of cyclobutane pyrimidine dimers (CPD), as determined by ELISA, in epidermis cells isolated from skin explants irradiated or not irradiated with 350 mJ/cm² UVB, following exposure to 0, 0.01, 0.02, 0.2 or 2 μg/cm² of SH-401 (n=3; * p<0.05 relative to non-irradiated vehicle control, # p<0.05 relative to irradiated vehicle control).

FIGS. 14A-14D presents histological images of skin explants subjected to 350 mJ/cm² UVB radiation (FIGS. 14B and 14D) or not subjected to UVB radiation (FIGS. 14A and 14C) with incubation with SH-401 (FIGS. 14C and 14D) or without incubation with SH-401 (FIGS. 14A and 14B) (Cont=control sample not exposed to either SH-401 or UVB; arrows in FIG. 14B emphasize altered features; all images are representative of samples in triplicate).

FIGS. 15A and 15B present bar graphs showing levels of pro-collagen (FIG. 15A) and MMP1 (FIG. 15B) in skin explants subjected to UVB radiation and the indicated concentrations of SH-101, or to radiation without SH-101 (UVB), as determined by ELISA (Cont=vehicle control sample not exposed to either SH-401 or UVB; * p<0.05 relative to vehicle control; # p<0.05 relative to UVB only; n=3).

FIG. 16 presents microscopic images of confluent HaCaT cells with an induced wound (between the two vertical lines) exposed to SH-101 or vehicle (ethanol) for 24 hours (Cont=control sample not exposed to either SH-401 or vehicle; all images are representative of samples in triplicate).

### DESCRIPTION OF SPECIFIC EMBODIMENTS OF THE INVENTION

The present invention, in some embodiments thereof, relates to photo-protection, and more particularly, but not exclusively, to UV-blocking composition which can be obtained from plant material.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have uncovered natural UV-absorbing material in sumac which acts as a particularly efficient agent for blocking harmful UV radiation (e.g., a sunscreen agent), and surprisingly exhibits additional beneficial effects on skin cells, such as anti-aging and wound healing effects.

While reducing the present invention to practice, the inventors further uncovered an efficient process for isolating specific UV-absorbing compounds, such as pentagalloyl glucose.

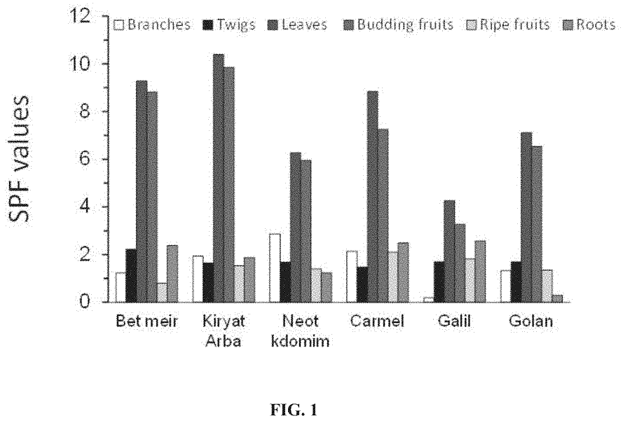

Referring now to the drawings, FIG. 1 shows that ethanol extracts of the leaves and budding fruits of sumac block harmful UV radiation to a greater extent than other portions of the plant.

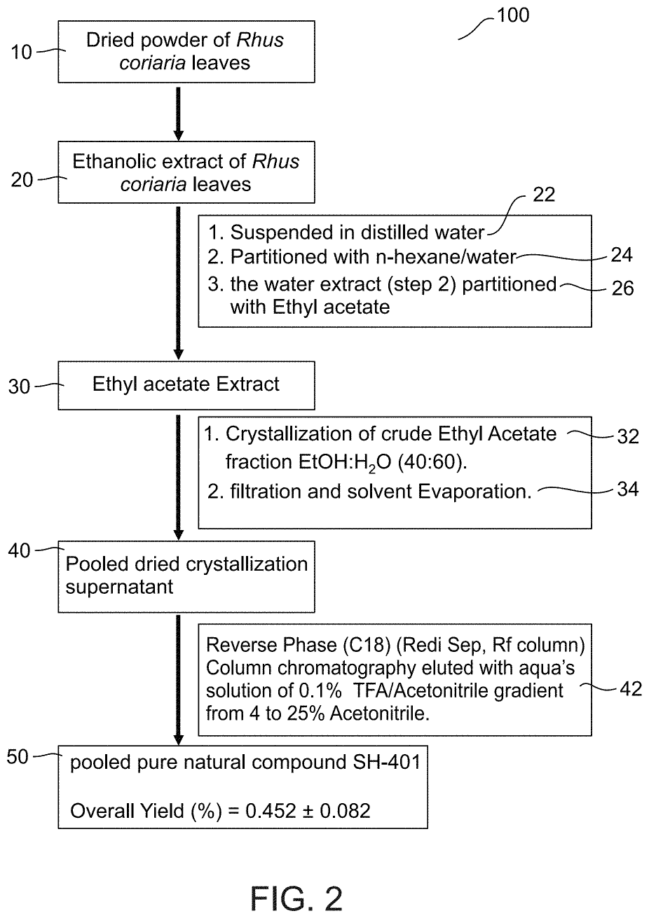

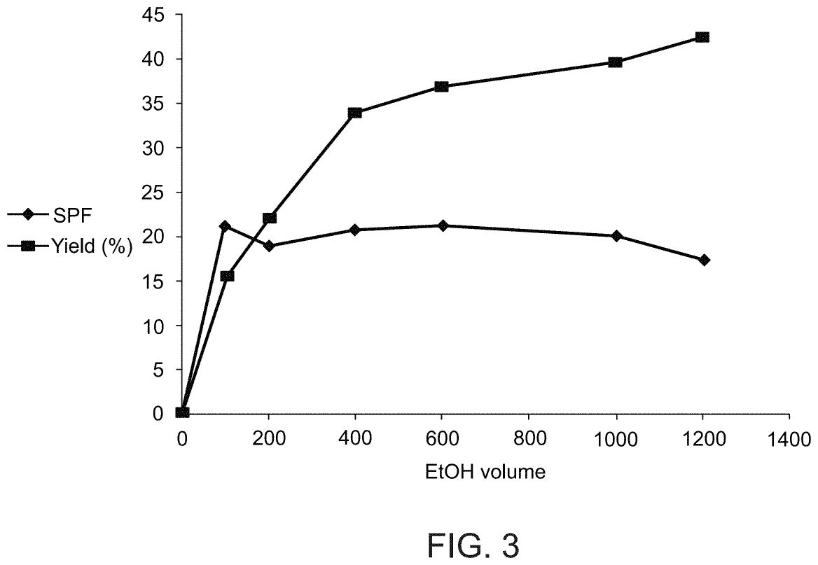

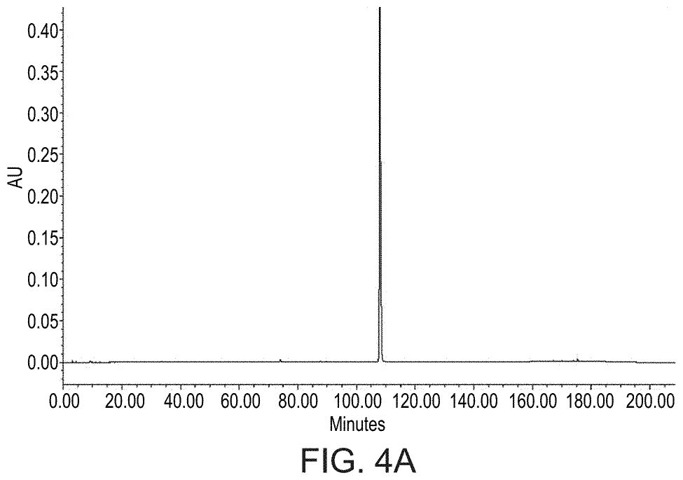

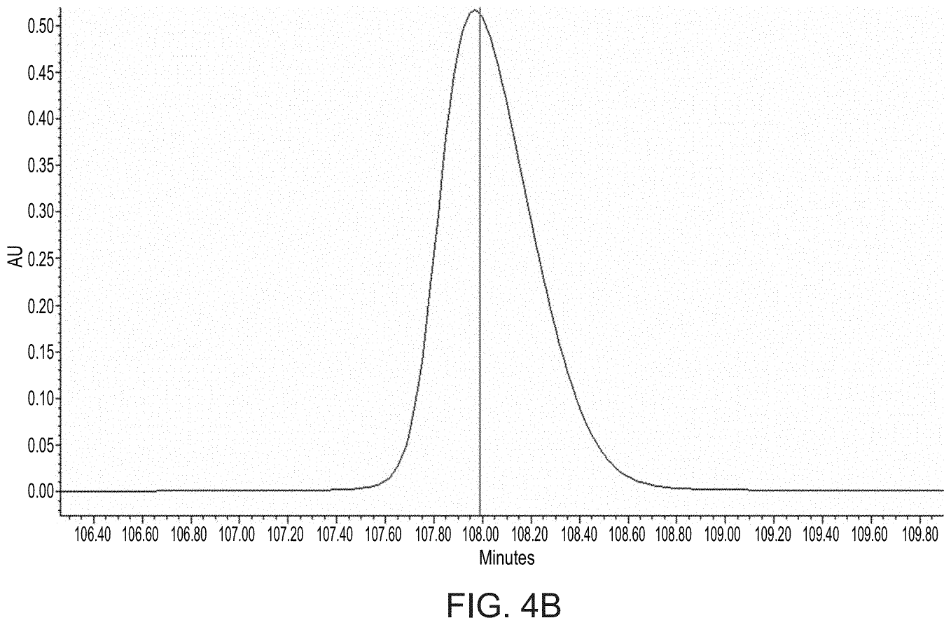

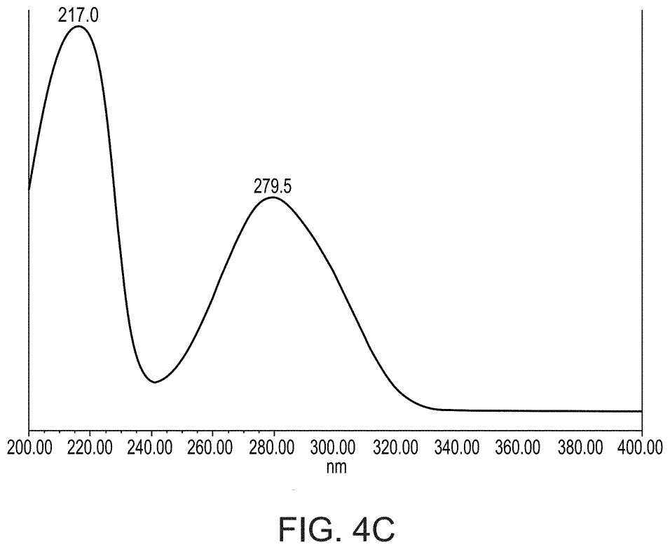

FIG. 2 shows a procedure for extracting an exemplary UV-absorbing substance (SH-101) from sumac leaves according to exemplary embodiments of the invention. FIGS. 4A-4B shows the purity and UV-absorption of SH-401. FIG. 3 shows the effect of ethanol volume on efficiency of extraction of UV-absorbing material.

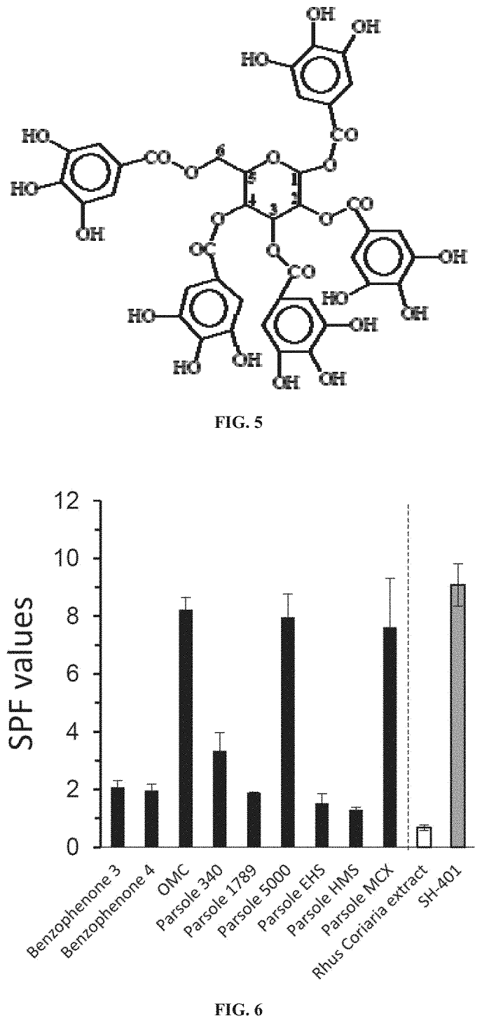

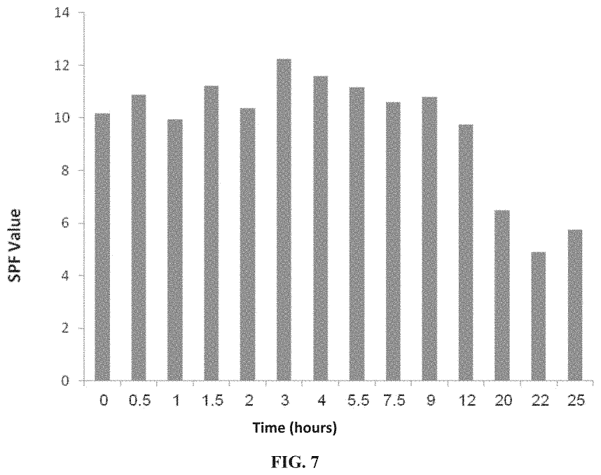

FIG. 5 shows the structure of SH-401 (pentagalloyl glucose), as determined by considerable spectroscopic evidence. FIG. 6 shows that the efficacy of SH-401 as a sunscreen agent (as measured by SPF value) compares favorably to commercial sunscreen agents. FIG. 7 shows that SH-401 is stable upon exposure to UV radiation.

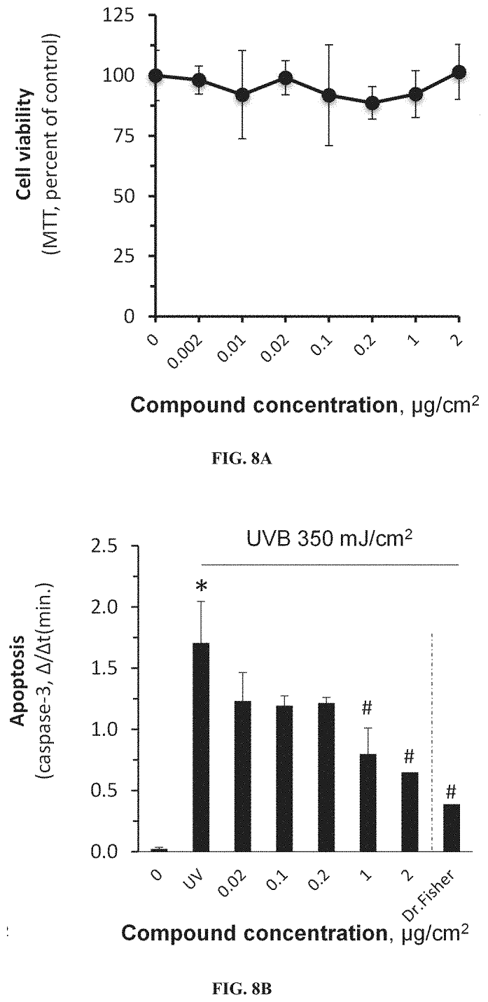

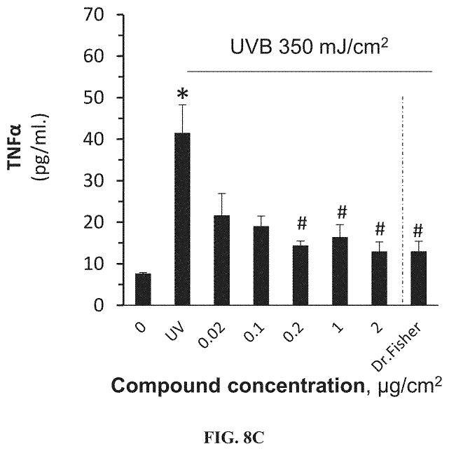

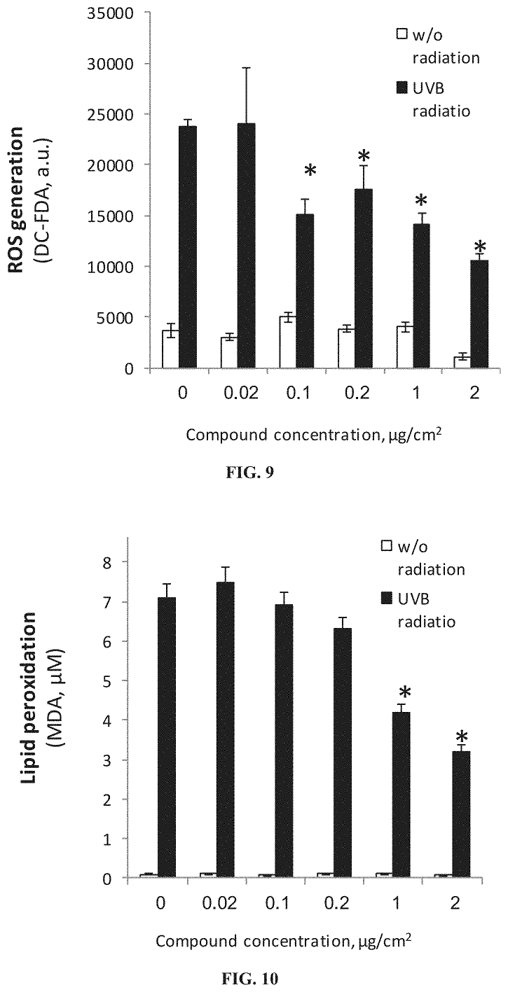

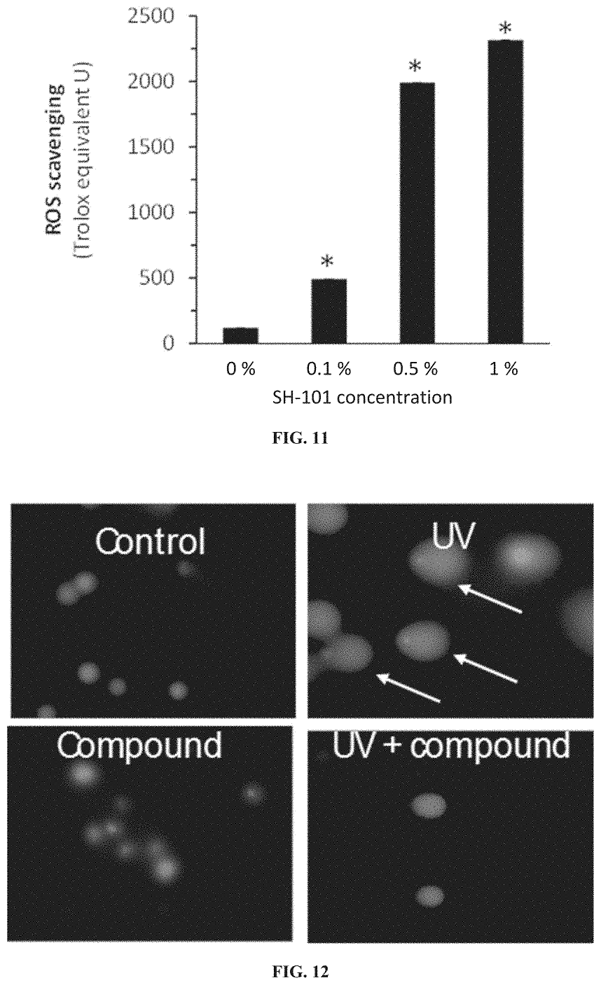

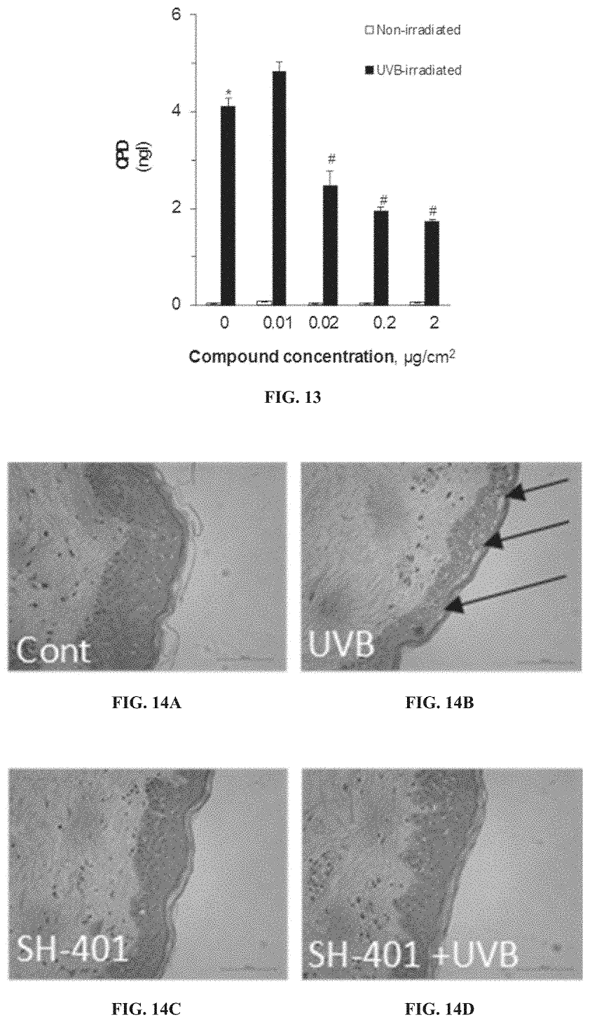

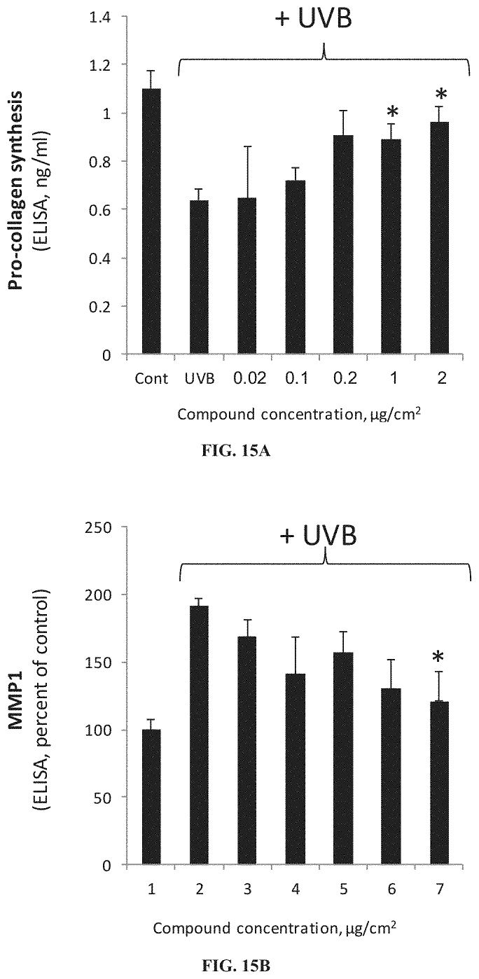

FIG. 8A shows that SH-401 is nontoxic to human skin cells, and FIGS. 8B, 8C and 14A-14D show that SH-401 protects human skin cells against UVB radiation. FIGS. 9 and 10 show that SH-401 reduces ROS (reactive oxygen species) generation and lipid peroxidation in human skin. FIG. 11 shows that SH-401 is an antioxidant. FIGS. 12 and 13 show that SH-401 reduces UV-induced DNA damage in skin cells. FIGS. 15A-15B show that SH-401 reverses UV-induced collagen degradation in skin.

Composition:

According to an aspect of embodiments of the invention, there is provided a composition comprising a UV-absorbing material and a dermatologically acceptable carrier, wherein the UV-absorbing material is extracted from sumac leaves and/or budding fruits.

Herein throughout, the term “UV-absorbing” refers to absorption of electromagnetic radiation in at least a portion of wavelengths in a range of from 200 nm to 500 nm. The subrange of 400 nm to 500 nm is also referred to herein as “blue light”, and the inclusion of absorption of such wavelengths within the scope of the term “UV-absorbing” is for the purposes of brevity.

In some of any of the respective embodiments described herein, the term “UV-absorbing” refers to absorption of at least a portion of wavelengths in a range of from 280 nm to 500 nm. In some such embodiment, the term “UV-absorbing” refers to absorption of at least a portion of wavelengths in a range of from 280 nm to 400 nm, wavelengths also referred to herein as “ultraviolet B” (UVB) (280-320 nm) and “ultraviolet A” (UVA) (320-400 nm). In some such embodiment, the term “UV-absorbing” refers to absorption of at least a portion of wavelengths in a range of from 280 nm to 320 nm (i.e., UVB). In some such embodiment, the term “UV-absorbing”refers to absorption of at least a portion of wavelengths in a range of from 290 nm to 320 nm (i.e., UVB).

In some of any of the respective embodiments described herein, the term “UV-absorbing” refers to absorption of at least a portion of wavelengths in a range of from 300 nm to 500 nm. In some such embodiment, the term “UV-absorbing” refers to absorption of at least a portion of wavelengths in a range of from 300 nm to 400 nm, wavelengths also referred to in the art as “near ultraviolet”.

In some of any of the respective embodiments described herein, the term “UV-absorbing” refers to absorption of blue light, that is, at least a portion of wavelengths in a range of from 400 nm to 500 nm. Materials absorbing such wavelengths may have a readily visible color, e.g., yellow.

In some embodiments, a concentration of the UV-absorbing material in the composition is at least 0.005 mg/ml. In some embodiments, a concentration of the UV-absorbing material in the composition is at least 0.01 mg/ml. In some embodiments, a concentration of the UV-absorbing material in the composition is at least 0.02 mg/ml. In some embodiments, a concentration of the UV-absorbing material in the composition is at least 0.05 mg/ml. In some embodiments, a concentration of the UV-absorbing material in the composition is at least 0.1 mg/ml. In some embodiments, a concentration of the UV-absorbing material in the composition is at least 0.2 mg/ml. In some embodiments, a concentration of the UV-absorbing material in the composition is at least 0.5 mg/ml. In some embodiments, a concentration of the UV-absorbing material in the composition is at least 1 mg/ml. In some embodiments, a concentration of the UV-absorbing material in the composition is at least 2 mg/ml. In some embodiments, a concentration of the UV-absorbing material in the composition is at least 5 mg/ml. In some embodiments, a concentration of the UV-absorbing material in the composition is at least 10 mg/ml. In some embodiments, a concentration of the UV-absorbing material in the composition is at least 20 mg/ml. In some embodiments, a concentration of the UV-absorbing material in the composition is at least 50 mg/ml. In some embodiments, a concentration of the UV-absorbing material in the composition is at least 100 mg/ml.

In some embodiments, a concentration of the UV-absorbing material in the composition is no more than 400 mg/ml, optionally no more than 200 mg/ml, optionally no more than 100 mg/ml, optionally no more than 50 mg/ml, optionally no more than 20 mg/ml, and optionally no more than 10 mg/ml.

In some embodiments, a concentration of the UV-absorbing material in the composition ranges from about 0.05 to about 10 mg/ml, or from about 10 mg/ml to about 50 mg/ml, or from about 50 to about 200 mg/ml, or from about 100 to about 400 mg/ml, including any intermediate values and subranges therebetween.

In some of any of the embodiments relating to a composition, the composition is a sunscreen composition.

Herein, the phrase “sunscreen composition” refers to a composition which, when applied as a thin layer on the skin, at least partially blocks or screens UV radiation (typically radiation in a range of from 290 to 320 nm, and optionally over a broader wavelength range, e.g., from 290 nm to 400 nm) from the sun by absorbing UV radiation and/or reflecting UV radiation. A sunscreen composition is optionally identified for use (e.g., in or on a packaging material in which the sunscreen composition is packaged) for blocking or screening UV radiation, or sunlight in general, and/or for minimizing harm (e.g., sunburns, cancer risks) associated with sunlight (e.g., by topical application).

In some of any of the embodiments described herein, a concentration of the UV-absorbing material in the composition is sufficient to absorb at least 90% of radiation at a wavelength of 300 nm over a path length of 1 mm. In some embodiments, the concentration is sufficient to absorb at least 90% of radiation at a wavelength of 300 nm over a path length of 3 mm. In some embodiments, the concentration is sufficient to absorb at least 90% of radiation at a wavelength of 300 nm over a path length of 0.1 mm. In some embodiments, the concentration is sufficient to absorb at least 90% of radiation at a wavelength of 300 nm over a path length of 0.03 mm. In some embodiments, the concentration is sufficient to absorb at least 90% of radiation at a wavelength of 300 nm over a path length of 0.01 mm.

As will be appreciated by the skilled person, absorption of at least 90% corresponds (according to the Beer-Lambert law) to absorbance of at least 1 (and absorption of at least 99% corresponds to absorbance of at least 2, and absorption of at least 99.9% corresponds to absorbance of at least 3), wherein the absorbance is a product of attenuation coefficient (an intrinsic property of a material), concentration, and path length. Thus, absorption of at least 90% over a path length of 1 mm corresponds to the product of attenuation coefficient and concentration being at least 1 mm⁻¹. Such a limitation therefore defines a concentration for a compound with a given absorption property. In the case of a mixture of compounds absorbing 300 nm radiation (e.g., in a UV-absorbing material described herein), the sum of the products of attenuation coefficient and concentration for each compound is at least 1 mm⁻¹.

As a given path length (e.g., 1 mm) defines a product of attenuation coefficient and concentration (e.g., being at least 1 mm⁻¹), it is to be appreciated that the absorption of the material may be measured for a different path length, for example, over a shorter path length in order to minimize scattering or absorption by other compounds in the composition. Thus, for example, an absorbance of at least 1 for a path length of 1 mm may optionally be determined in practice as an absorbance of at least 10 (as defined herein and in the art) for a path length of 10 mm, an absorbance of 0.1 (as defined herein and in the art) for a path length of 0.1 mm, and so forth.

In some of any of the embodiments described herein, the UV-absorbing material comprises at least one compound having a plurality of galloyl (i.e., 3,4,5-trihydroxybenzoyl) substituents, for example, from 2 to 10 galloyl substituents, or from 4 to 6 (e.g., 5) galloyl substituents. In some embodiments, the at least one compound have a carbohydrate moiety, for example, a glucose moiety, with a plurality of galloyl substituents. The carbohydrate moiety may optionally be a sugar (e.g., hexose) moiety, such as a D-glucose moiety, or a quinic acid moiety.

In some of any of the respective embodiments described herein, galloyl substituents are attached to another moiety via an ester bond, that is, the galloyl is attached to an oxygen atom of the other moiety.

The galloyl substituents may optionally each be attached to a backbone moiety (e.g., a carbohydrate moiety), or alternatively, some galloyl substituents may optionally be attached directly to the backbone moiety and some galloyl substituents may be attached to other galloyl substituents. In some such embodiments, each of the galloyl substituents of the backbone moiety (e.g., a glucose moiety) is in a form of galloyl per se, or galloyl substituted by galloyl (e.g., 3,4-dihydroxy-5-[(3,4,5-trihydroxybenzoyl)oxy]benzoyl). In some such embodiments, each of the galloyl substituents of the backbone moiety (e.g., a glucose moiety) is in a form of galloyl per se.

As exemplified herein, 1,2,3,4,6-pentagalloyl glucose (comprising 5 galloyl moieties attached directly to different oxygen atoms of glucose) may be extracted from sumac with a high degree of efficiency.

In some of any of the respective embodiments, a concentration of 1,2,3,4,6-pentagalloyl glucose in the UV-absorbing material is least 80 weight percents. In some embodiments, the concentration of the 1,2,3,4,6-pentagalloyl glucose is at least 90 weight percents. In some embodiments, the concentration of the 1,2,3,4,6-pentagalloyl glucose is at least 95 weight percents. In some embodiments, the concentration of the 1,2,3,4,6-pentagalloyl glucose is at least 98 weight percents. In some embodiments, the concentration of the 1,2,3,4,6-pentagalloyl glucose is at least 99 weight percents.

In some of any of the embodiments described herein, the UV-absorbing material according to any of the respective embodiments described herein is 1,2,3,4,6-pentagalloyl glucose isolated from sumac leaves and/or budding fruits (e.g., according to any of the embodiments described herein relating to isolation of 1,2,3,4,6-pentagalloyl glucose from sumac). In some embodiments, the concentration of the 1,2,3,4,6-pentagalloyl glucose is at least 0.005 mg/ml (according to any of the respective embodiments described herein).

Herein throughout, the term “sumac” refers to Rhus spp., that is, any plant belonging to the genus Rhus. Examples of sumac include, without limitation, Rhus chinensis (a.k.a. Chinese sumac), Rhus delavayi, Rhus hypoleuca, Rhus punjabensis (a.k.a. Punjab sumac), Rhus taitensis, Rhus sanwicensis, Rhus coriaria (a.k.a. tanner's sumac, Sicilian sumac, or elm-leaved sumac), Rhus aromatica (a.k.a. fragrant sumac), Rhus copallinum (a.k.a. winged sumac or shining sumac), Rhus glabra (a.k.a. smooth sumac), Rhus lanceolata (a.k.a. prairie sumac), Rhus michauxii (a.k.a. Michaux's sumac), Rhus typhina (a.k.a. staghorn sumac), Rhus choriophylla (a.k.a. Mearn's sumac), Rhus integrifolia (a.k.a. lemonade sumac), Rhus kearneyi (a.k.a. Kearney sumac), Rhus microphylla (a.k.a. desert sumac or littleleaf sumac), Rhus ovata (a.k.a. sugar sumac), Rhus trilobata (a.k.a. skunkbush sumac), Rhus virens (a.k.a. evergreen sumac), and Rhus muelleri (a.k.a. Muller's sumac). In exemplary embodiments, the sumac is Rhus coriaria.

In some of any of the embodiments described herein, the UV-absorbing material is obtainable by a process described herein, according to any of the respective embodiments.

Process:

According to an aspect of embodiments of the invention there is provided a process for obtaining a UV-absorbing material (e.g., a UV-absorbing material according to any of the respective embodiments described herein) from sumac. The process comprising contacting sumac leaves and/or budding fruits with a water-miscible organic solvent (e.g., thereby obtaining a UV-absorbing material as an extract). In some embodiments, the process further comprises removing the water-miscible organic solvent, for example, by evaporation of the solvent.

The sumac leaves and/or budding fruits are optionally dried prior to contacting with the water-miscible organic solvent, for example, by exposure to air (optionally dry air) and/or by mild heating (e.g., at a temperature below 100° C., or below 75° C., or below 50° C.).

In some of any of the respective embodiments described herein, the water-miscible organic solvent comprises an alcohol (optionally two or more alcohols), comprising one —OH group or more than one —OH group (e.g., ethylene glycol, propanediol, butanediol, pentanediol, and/or glycerin); and/or a ketone (e.g., acetone). In some embodiments, the water-miscible organic solvent comprises a C₁₋₄-alcohol (e.g., t-butyl alcohol, 1-propanol, isopropanol, ethanol and/or methanol), acetone and/or glycerin. Ethanol is an exemplary water-miscible organic solvent according to some embodiments.

In some of any of the respective embodiments described herein, contacting the sumac leaves and/or budding fruits with a water-miscible organic solvent comprises extraction. In some such embodiments, the extraction is effected using a Soxhlet extractor.

Herein, the term “Soxhlet extractor” refers to an apparatus configured for recycling a solvent used for extraction, by contacting the solvent with a source (e.g., sumac) so as to extract a material, evaporating solvent used to extract a material, and condensing the solvent vapor such that the vapor returns to contact the source, thereby further extracting material. The term “Soxhlet extractor” encompasses various specific designs, including what is also known in the art as a “Kumagawa extractor”.

In embodiments relating to a Soxhlet extractor, a volatile water-miscible organic solvent is particularly suitable, for example, a solvent having a boiling point (at atmospheric pressure) of no more than 100° C.

In some of any of the respective embodiments described herein, the amount of water-miscible organic solvent contacted with the sumac is at least 5 ml solvent per gram (dry weight) of sumac, optionally at least 10 ml solvent per gram (dry weight) of sumac, and optionally at least 15 ml solvent per gram (dry weight) of sumac. In some such embodiments, the solvent comprises ethanol.

In some of any of the respective embodiments described herein, the amount of water-miscible organic solvent contacted with the sumac is no more than 40 ml solvent per gram (dry weight) of sumac, optionally no more than 30 ml solvent per gram (dry weight) of sumac, and optionally no more than about 20 ml solvent per gram (dry weight) of sumac. In some such embodiments, the solvent comprises ethanol.

In some of any of the respective embodiments described herein, the amount of water-miscible organic solvent contacted with the sumac is in a range of from 5 to 40 ml solvent per gram (dry weight) of sumac, and optionally from 10 to 30 ml solvent per gram (dry weight) of sumac. As exemplified herein, about 20 ml water-miscible organic solvent per gram (dry weight) of sumac can provide efficient extraction. In some embodiments, the solvent comprises ethanol.

In some of any of the respective embodiments described herein, the process further comprises partitioning a UV-absorbing material (e.g., in a form of an extract obtained using a water-miscible organic solvent, according to any of the respective embodiments described herein) in a polar solvent and a nonpolar solvent, and collecting the fraction which partitions into the polar solvent, for example, to thereby obtain a more pure (more efficiently absorbing) UV-absorbing material.

Herein, the term “partitioning” refers to contacting a material with two immiscible liquids, allowing different portions of the material to pass (i.e., “partition”) to the different phases, e.g., based on different affinities and/or solubilities of different components of the material in each of the two liquids. One or both of the phases may optionally be separated from the other phase, thereby collecting the portion which partitioned into that phase.

In some of any of the respective embodiments described herein, the nonpolar solvent comprises one or more hydrocarbons, and optionally one or more aliphatic hydrocarbons. In some embodiments, the nonpolar solvent comprises one or more alkanes, for example, one or more alkanes having from 5 to 16 carbon atoms. Hexane is an exemplary nonpolar solvent according to some embodiments.

In some of any of the respective embodiments described herein, the polar solvent comprises water, for example, at least 80 weight percents water, or at least 90 weight percents water, or at least 95 weight percents water, or at least 98 weight percents water, or at least 99 weight percents water. In exemplary embodiments, the polar solvent consists essentially of water.

In some of any of the respective embodiments described herein, the nonpolar solvent is an aliphatic hydrocarbon, and the polar solvent consists essentially of water.

Without being bound by any particular theory, it is believed that the nonpolar solvent separates considerable amounts of other plant-derived substances, e.g., chlorophyll, from the UV-absorbing material which partitions into the polar solvent. It is further believed that suitable ratios of polar and nonpolar solvents (e.g., as described herein) may enhance the proportion of separated plant-derived substances and/or minimize the proportion of UV-absorbing material which goes to waste.

In some of any of the respective embodiments described herein, a ratio of nonpolar solvent to water used in partitioning is at least 0.05 ml solvent per ml of water, optionally at least 0.1 ml solvent per ml of water, optionally at least 0.15 ml solvent per ml of water, and optionally at least 0.25 ml solvent per ml of water. In some such embodiments, the solvent comprises hexane.

In some of any of the respective embodiments described herein, a ratio of nonpolar solvent to water used in partitioning is no more than 1 ml solvent per ml of water, optionally no more than 0.6 ml solvent per ml of water, optionally no more than 0.4 ml solvent per ml of water, and optionally no more than about 0.25 ml solvent per ml of water. In some such embodiments, the solvent comprises hexane.

In some of any of the respective embodiments described herein, a ratio of nonpolar solvent to water used in partitioning is in a range of from 0.05 to 1 ml solvent per ml of water, optionally in a range of from 0.1 to 0.6 ml solvent per ml of water, optionally in a range of from 0.15 to 0.4 ml solvent per ml of water, and optionally about 0.25 ml solvent per ml of water. In some such embodiments, the solvent comprises hexane.

In some of any of the respective embodiments described herein, the process further comprises partitioning a UV-absorbing material in water and a water-immiscible polar organic solvent, and collecting the fraction which partitions into the water-immiscible polar organic solvent, for example, to thereby obtain a more pure (more efficiently absorbing) UV-absorbing material. The UV-absorbing material is optionally in a form of a fraction which partitioned into a polar solvent (rather than a nonpolar solvent), according to any of the respective embodiments described herein.

Examples of water-immiscible polar organic solvents include, without limitation, esters, such as C₁₋₄-alkyl acetate (e.g., methyl acetate, ethyl acetate, isopropyl acetate, and butyl acetate) and propylene carbonate; alcohols, such as 1-butanol, 2-butanol, isobutanol, amyl alcohol, isoamyl alcohol, octanol, and cyclohexanol; ketones, such as 2-butanone, methyl isobutyl ketone, acetophenone and cyclohexanone; aldehydes, such as furfuraldehyde; amines, such as aniline; polar chlorinated solvents, such as dichloromethane and 1,2-dichloroethane; carbon disulfide; and nitro compounds, such as nitromethane, nitropropane and nitrobenzene. Ethyl acetate is an exemplary water-immiscible polar organic solvent according to some embodiments.

In some of any of the respective embodiments described herein, a ratio of water-immiscible polar organic solvent to water used in partitioning is at least 0.3 ml solvent per ml of water, optionally at least 0.6 ml solvent per ml of water, optionally at least 1 ml solvent per ml of water, and optionally at least 1.5 ml solvent per ml of water. In some such embodiments, the solvent comprises ethyl acetate.

In some of any of the respective embodiments described herein, a ratio of water-immiscible polar organic solvent to water used in partitioning is no more than 7.5 ml solvent per ml of water, optionally no more than 4 ml solvent per ml of water, optionally no more than 2.5 ml solvent per ml of water, and optionally no more than about 1.5 ml solvent per ml of water. In some such embodiments, the solvent comprises ethyl acetate.

In some of any of the respective embodiments described herein, a ratio of water-immiscible polar organic solvent to water used in partitioning is in a range of from 0.3 to 7.5 ml solvent per ml of water, optionally in a range of from 0.6 to 4 ml solvent per ml of water, optionally in a range of from 1 to 2.5 ml solvent per ml of water, and optionally at about 1.5 ml solvent per ml of water. In some such embodiments, the solvent comprises ethyl acetate.

In some of any of the respective embodiments described herein, the process further comprises effecting crystallization of the UV-absorbing material, for example, to thereby obtain a more pure UV-absorbing material. Crystallization may optionally be effected by any suitable technique known in the art. In some embodiments, crystallization is effected by contacting the UV-absorbing material (e.g., in a form of a fraction which partitioned into a water-immiscible polar organic solvent, according to any of the respective embodiments described herein) with a solvent comprising water.

The solvent comprising water may optionally be, for example, a mixture of water and one or more water-miscible organic solvents (according to any of the respective embodiments described herein. In some of any of the respective embodiments, the solvent comprising water comprises water and an alcohol, for example, at a concentration of in a range of from about 20 to about 80 weight percents (e.g., an alcohol:water weight ratio of from 20:80 to 80:20 alcohol:water), or from about 30 to about 60 weight percents (e.g., an alcohol:water weight ratio of from 30:70 to 60:40), or about 40 weight percents (e.g., an alcohol:water weight ratio of 40:60). Ethanol/water (e.g., at a 40:60 weight ratio) is an exemplary solvent comprising water, for effecting crystallization.

Following crystallization, crystals of the UV-absorbing material may optionally be separated from solvent by filtration and/or evaporation of solvent. An isolated UV-absorbing material at a relatively high degree of purity may thus be obtained.

In some of any of the respective embodiments described herein, a UV-absorbing material obtained according to any of the respective embodiments described herein (optionally comprising crystallization as the previous step) is further purifying by column chromatography, for example, by identifying a fraction exiting a column by UV absorption. In some embodiments, the stationary phase is hydrophobic. In exemplary embodiments, a carbon chain (octadecyl, or C18)-bonded silica is used as a stationary phase. A gradient of aqueous solution (e.g., 0.1% trifluoroacetic acid) and acetonitrile is an exemplary mobile phase.

In some of any of the embodiments described herein relating to a process for obtaining a UV-absorbing material comprising 1,2,3,4,6-pentagalloyl glucose, the 1,2,3,4,6-pentagalloyl glucose in the UV-absorbing material is at a relatively high degree of purity, for example, least 95 weight percents of the UV-absorbing material. In some embodiments, the purity of the 1,2,3,4,6-pentagalloyl glucose is at least 98 weight percents. In some embodiments, the purity of the 1,2,3,4,6-pentagalloyl glucose is at least 99 weight percents. In some embodiments, the purity of the 1,2,3,4,6-pentagalloyl glucose is at least 99.5 weight percents. In some embodiments, the purity of the 1,2,3,4,6-pentagalloyl glucose is at least 99.8 weight percents.

According to an aspect of embodiments of the invention there is provided a UV-absorbing material obtained according to the process described herein, according to any of the respective embodiments.

Formulation and Uses:

As mentioned hereinabove, the UV-absorbing material according to any of the embodiments presented herein can be utilized as a part of a composition comprising a dermatologically acceptable carrier.

Such a composition may optionally be any composition intended for topical use.

As further mentioned hereinabove, the composition is optionally a sunscreen composition, that is, intended for use in blocking or screening UV radiation (e.g., in sunlight). It is to be appreciated that a sunscreen composition (according to any of the respective embodiments described herein) may optionally have a primary intended use other than as a sunscreen (e.g., a cosmetic and/or pharmaceutical use), with the sunscreen activity being an auxiliary activity of the composition.

Alternatively or additionally, the composition (according to any of the respective embodiments described herein) is optionally a pharmaceutical composition and/or cosmetic composition (e.g., a composition included in a cosmetic product). In some such embodiments, the composition is identified for use in treating skin aging or a wound, e.g., a pharmaceutical composition for use in promoting wound healing. In some embodiments, the cosmetic composition is a skin rejuvenation composition and/or a peeling composition, e.g., identified for use in rejuvenating skin or in skin peeling.

Herein, the terms “cosmetic” and “cosmetic composition” and “cosmetic product” refer to topical substances or products (articles of manufacturing) that are utilized for aesthetical purposes. Cosmetic compositions optionally include substances that further exhibit pharmaceutical activity so as to facilitate providing the desired aesthetical effect.

Cosmetic compositions or products in which the active ingredients described herein can be beneficially utilized include, for example, make ups, gels, lacquers, eye shadows, lip glosses, lipsticks, and the like.

Herein, the terms “pharmaceutical” and “pharmaceutically” refer to any compound and/or composition intended for beneficially altering a condition and/or behavior of at least a portion of the body (e.g., skin), including cosmetically altering, e.g., the skin. It is to be appreciated that such a definition may be broader than the use of such terms by regulatory agencies, which may exclude, for example, cosmetic effects from the scope of the terms.

The effects of a pharmaceutical or cosmetic composition may optionally be associated with protection against damage induced by UV radiation, which effect is optionally, but not necessarily, mediated at least in part by reducing an amount of UV radiation which reaches the skin.

Alternatively or additionally, a beneficial effect of a pharmaceutical or cosmetic composition may be associated with protection against damage induced by UV radiation by a mechanism other than UV radiation reduction (for example, by an antioxidant effect), and/or be associated with treatment of damage not associated with UV radiation, for example, wounds (e.g., by promoting healing of wounds not associated with UV radiation).

According to another aspect of embodiments of the invention, there is provided a use of a UV-absorbing material derived from sumac or a composition comprising UV-absorbing material derived from sumac (according to any of the respective embodiments described herein) in the manufacture of a medicament, e.g., a medicament for treating skin aging or a wound.

According to another aspect of embodiments of the invention, there is provided a method of treating skin aging and/or a wound in a subject in need thereof, the method comprising topically administering to the subject a composition comprising UV-absorbing material derived from sumac (according to any of the respective embodiments described herein).

Herein, the term “dermatologically acceptable carrier” refers to a carrier or a diluent that does not cause significant irritation to an organism when applied to the skin of the organism and does not abrogate the biological activity and properties of the administered compound.

Compositions for use in accordance with embodiments of the present invention thus may be formulated in conventional manner using one or more carriers comprising excipients and auxiliaries, which facilitate processing of the abovementioned compounds into preparations which, can be used cosmetically and/or pharmaceutically.

Herein the term “excipient” refers to an inert substance added to a composition to further facilitate administration of an active ingredient, e.g., a UV-absorbing material according to any of the respective embodiments described herein. Examples of suitable solid or gel phase carriers or excipients include, but are not limited to, calcium carbonate, calcium phosphate, various sugars, starches, cellulose derivatives, gelatin and polymers such as polyethylene glycols.

Techniques for formulation and administration of active ingredients may be found in “Remington's Pharmaceutical Sciences” Mack Publishing Co., Easton, Pa., latest edition, which is incorporated herein by reference.

Compositions described herein according to various embodiments of the present invention may be manufactured by processes well known in the art, e.g., by means of conventional mixing, dissolving, granulating, levigating, emulsifying, encapsulating, entrapping or lyophilizing processes.

By selecting the appropriate carrier and optionally other ingredients that can be included in the composition, as is detailed herein, the compositions described herein may be formulated into any form suitable for topical application. Hence, the compositions can be, for example, in a form of a cream, an ointment, a paste, a gel, a lotion, and/or a soap.

Ointments are semisolid preparations, typically based on vegetable oil (e.g., shea butter and/or cocoa butter), petrolatum or petroleum derivatives. As with other carriers or vehicles, an ointment base should be inert, stable, nonirritating and non-sensitizing.

Lotions are preparations that may to be applied to the skin without friction. Lotions are typically liquid or semiliquid preparations with a water or alcohol base, for example, an emulsion of the oil-in-water type. Lotions are typically preferred for treating large areas (e.g., as is frequently desirable for sunscreen compositions), due to the ease of applying a more fluid composition.

Creams are viscous liquids or semisolid emulsions, either oil-in-water or water-in-oil. Cream bases typically contain an oil phase, an emulsifier and an aqueous phase. The oil phase, also called the “lipophilic” phase, optionally comprises petrolatum and/or a fatty alcohol such as cetyl or stearyl alcohol. The aqueous phase optionally contains a humectant. The emulsifier in a cream formulation is optionally a nonionic, anionic, cationic or amphoteric surfactant.

Herein, the term “emulsion” refers to a composition comprising liquids in two or more distinct phases (e.g., a hydrophilic phase and a lipophilic phase). Non-liquid substances (e.g., dispersed solids and/or gas bubbles) may optionally also be present.

As used herein and in the art, a “water-in-oil emulsion” is an emulsion characterized by an aqueous phase which is dispersed within a lipophilic phase.

As used herein and in the art, an “oil-in-water emulsion” is an emulsion characterized by a lipophilic phase which is dispersed within an aqueous phase.

Pastes are semisolid dosage forms which, depending on the nature of the base, may be a fatty paste or a paste made from a single-phase aqueous gel. The base in a fatty paste is generally petrolatum, hydrophilic petrolatum, and the like. The pastes made from single-phase aqueous gels generally incorporate carboxymethylcellulose or the like as a base.

Gel formulations are semisolid, suspension-type systems. Single-phase gels optionally contain organic macromolecules distributed substantially uniformly throughout the carrier liquid, which is typically aqueous; but also, preferably, contains a non-aqueous solvent, and optionally an oil. Preferred organic macromolecules (e.g., gelling agents) include crosslinked acrylic acid polymers such as the family of carbomer polymers, e.g., carboxypolyalkylenes, that may be obtained commercially under the trademark Carbopol®. Other types of preferred polymers in this context are hydrophilic polymers such as polyethylene oxides, polyoxyethylene-polyoxypropylene copolymers and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

A composition formulated for topical administration may optionally be present in a patch, a swab, a pledget, and/or a pad.

Dermal patches and the like may comprise some or all of the following components: a composition to be applied (e.g., as described herein); a liner for protecting the patch during storage, which is optionally removed prior to use; an adhesive for adhering different components together and/or adhering the patch to the skin; a backing which protects the patch from the outer environment; and/or a membrane which controls release of a drug to the skin.

According to optional embodiments, the composition is stable (e.g., devoid of substantial chemical changes and/or phase separation) at room temperature (e.g., 20° C.) for at least 2 weeks, optionally at least 1 month, optionally at least 2 months, optionally at least 6 months, and optionally at least 1 year.

Pharmaceutical compositions, cosmetic compositions and sunscreen compositions suitable for use in context of embodiments of the present invention include compositions wherein the active ingredients are contained in an effective amount for achieving the respective intended purpose.

Determination of an effective amount for a given purpose is well within the capability of those skilled in the art, especially in light of the detailed disclosure provided herein.

The amount of a pharmaceutical or cosmetic composition and/or active ingredient in a pharmaceutical or cosmetic composition to be administered may be dependent on the subject being treated, the severity of the affliction, the manner of administration, the judgment of a physician prescribing a pharmaceutical composition, etc.

The amount of sunscreen composition and/or active ingredient (e.g., UV-absorbing material according to any of the respective embodiments described herein) to be administered may be dependent on factors such as the degree of sunlight to be protected against, the sensitivity of the subject to sunlight (e.g., as affected by skin pigmentation), and whether full protection is desirable or not (e.g., moderate UV exposure may be desired to induce tanning).

The degree of UV-blocking activity afforded by a sunscreen composition (correlating, for example, to active ingredient concentration) may optionally be expressed according to techniques known in the art, for example, expressed quantitatively as a sun protection factor (SPF) value.

The composition according to any of the respective embodiments described herein may optionally further comprise additional active ingredients suitable for providing an intended effect of a composition, e.g., as described herein. Such an additional active ingredient may be, for example, a sunscreen agent, UV-absorbing agent, antioxidant, skin-care agent and/or agent for treating a condition described herein.

For example, additional active ingredients suitable for use in blocking or UV radiation (e.g., for use in a sunscreen composition) include, without limitation, benzophenones (e.g., benzophenone-1, benzophenone-2, benzophenone-3, benzophenone-4, benzophenone-5, benzophenone-6, benzophenone-7, benzophenone-8, benzophenone-9, benzophenone-10, and diethylamino hydroxybenzoyl hexyl benzoate), p-aminobenzoic acid and derivatives thereof, such as N-alkyl-substituted derivatives and/or esters thereof (e.g., isopentyl N-dimethyl-p-aminobenzoate and octyl N-dimethyl-p-aminobenzoate), avobenzone, bemotrizinol, bisoctrizole, 3-(4-methylbenzylidene)-camphor, drometrizole trisiloxane, ecamsule, ethylhexyl triazone, menthyl anthranilate, octocrylene, iscotrizinol, methoxycinnamate or derivatives (e.g., esters) thereof (e.g., isopentyl 4-methoxycinnamate, octyl 4-methoxycinnamate, and cinoxate), polysilicone-15, salicylic acid and salts (e.g., trolamine salicylate) or derivatives (e.g., esters) thereof (e.g., homosalate and octyl salicylate), and inorganic substances such as TiO₂ and/or ZnO.

In some embodiments, an additional active (UV-blocking) ingredient is TiO₂ and/or ZnO, e.g., such that synthetic organic agents may optionally be avoided.

Additional active ingredients suitable for use in a composition for treating a wound and/or promoting wound healing include, without limitation, skin soothing and/or healing agents such as panthenol and derivatives thereof (e.g., ethyl panthenol), aloe vera, pantothenic acid and its derivatives, allantoin, bisabolol, and dipotassium glycyrrhizinate.

Antioxidants suitable for use as additional active ingredients (e.g., for reducing UV-induced or non-UV-induced damage to skin) in a composition described herein include, without limitation, ascorbic acid, butylated hydroxyanisole, butylated hydroxytoluene, carotenes and carotenoids (e.g., alpha-carotene, beta-carotene, canthaxanthin, cryptoxanthin, lutein, lycopene, zeaxanthin, and vitamin A), curcumin, eugenol, flavonoids (e.g., flavones, isoflavones, flavanols, flavonols, flavanones, stilbenoids, anthocyanins), glutathione, propyl gallate, tertiary butylhydroxyquinone, tocopherols (e.g., vitamin E), uric acid, and antioxidant enzymes (e.g., thioredoxin, catalase and superoxide dismutase).

The compositions described herein may also include additional components which are added, for example, in order to enrich the compositions with fragrance and nutrition factors (e.g., skin or hair nutrition factors).

Such components are selected suitable for topical use on a human without inducing toxicity, incompatibility, instability, allergic response, and the like within the scope of sound medical judgment. In addition, such optional components are useful provided that they do not unacceptably alter the benefits of the active ingredient(s) of the invention.

The CTFA Cosmetic Ingredient Handbook, Second Edition (1992) describes a wide variety of non-limiting cosmetic ingredients commonly used in the skin care industry, which are suitable for use in the compositions of the present invention. Examples of these ingredient classes include: abrasives; absorbents; aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate); anti-acne agents; anti-caking agents; antifoaming agents; antimicrobial agents; antioxidants (e.g., for use in preserving the composition, rather than as an active ingredient); binders; biological additives; buffering agents; bulking agents; chelating agents; chemical additives; colorants; cosmetic astringents; cosmetic biocides; denaturants; drug astringents; external analgesics; film formers or materials, e.g., polymers, for aiding the film-forming properties and substantivity of the composition (e.g., copolymer of eicosene and vinyl pyrrolidone); opacifying agents; pH adjusters; propellants; reducing agents; sequestrants; skin-conditioning agents (e.g., humectants, including miscellaneous and occlusive); skin treating agents; thickeners; and vitamins and derivatives thereof.

As compositions according to some embodiments described herein are for utilization in vivo, the composition is preferably of high purity and substantially free of potentially harmful contaminants, e.g., at least National Food (NF) grade, generally at least analytical grade, and preferably at least pharmaceutical grade. To the extent that a given compound must be synthesized prior to use, such synthesis or subsequent purification shall preferably result in a product that is substantially free of any potentially contaminating toxic agents that may have been used during the synthesis or purification procedures.

Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack. The pack or dispenser device may be accompanied by instructions for administration. The pack or dispenser may also be accompanied by a notice associated with the container in a form prescribed by a governmental agency regulating the manufacture, use or sale of cosmetics and/or pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions according to any of the embodiments of the invention may also be prepared, placed in an appropriate container, and labeled for treatment and/or protection of skin (e.g., according to any of the embodiments described herein).

Thus, according to some embodiments of the present invention, the pharmaceutical compositions described herein are packaged in a packaging material and identified in print, in or on the packaging material, for use in treating a condition described herein in a subject in need thereof.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Materials:

DMEM (Dulbecco's modified Eagle medium, supplemented with 100 units/ml penicillin and 100 μg/ml streptomycin) was obtained from Biological Industries (Israel).

DPPH (diphenylpicrylhydrazyl) was obtained from Sigma Aldrich.

Ethanol was obtained from Mercury Scientific & Industrial Products Ltd.

Methanol was obtained from Mercury Scientific & Industrial Products Ltd.

MTT (3-(4,5-dimethylthiazol-2-yl)-2,5-diphenyltetrazolium bromide) was obtained from Sigma Aldrich.

Protection Factor Calculation:

Absorption spectra were used to calculate SPF (sun protection factor) values using the Mansur equation, according to procedures described by Dutra et al. [Braz J Pharma Sci 2004, 40:381-385], using OptLab™ X software (Ascanis).

High-Performance Liquid Chromatography:

An HPLC (high-performance liquid chromatography) chromatogram was obtained by diluting a sample to a concentration of 0.01 mg/ml in a 95:5 mixture of double distilled water:methanol, loading 10 μl onto an RP-18 column of a Waters HPLC apparatus with a photodiode array detector, and using a gradient of from 96% aqueous solution of trifluoroacetic acid (0.1%) to 100% acetonitrile with a flow rate of 1 ml/minute.

Testing on Human Skin Explants:

Skins were obtained with permission from 30-60 year-old healthy women undergoing aesthetic abdomen surgery, after signing an informed consent. A mechanical skin press was used to section the skin to 0.8×0.8 cm² pieces, according to procedures described in Portugal-Cohen et al. [Exp Dermatol 2011, 20:749-755]. The skin explants were maintained in an air/liquid interface, dermal side submerged in the medium, according to procedures described in Portugal-Cohen et al. [Exp Dermatol 2011, 20:749-755] and Cohen et al. [Dead Sea and Arava Studies 2015, 7:66-74]. The human skin explants were laid in 6-well culture plates containing DMEM (Dulbecco's modified Eagle medium) supplemented with 100 units/ml penicillin and 100 μg/ml streptomycin), dermal side down in the medium and epidermis up. All samples were used after an overnight recovery.

### Example 1

### Screening of Plants for UV-Blocking Ability

In order to investigate UVB blocking properties of various plants, a large-scale screening of traditionally-used herbs which grow in the Judean Desert was performed. The leaves, twigs, branches, fruits, and roots of each tested plant were collected, dried, ground and extracted separately in ethanol, water, chloroform or propylene glycol methyl ether acetate (PMA) using a Soxhlet system. After evaporation of the solvent in vacuum, 0.1 gram of the crude extract was re-suspended in an equal volume of solvent using sonication and diluted to a fixed concentration for analysis of the UV absorbance spectrum, using a Cary™ 60 dual-beam spectrophotometer without integrating sphere (Agilent).

Preliminary results (data not shown) indicated that the ethanol leaf extracts in most of the tested plant species exhibited the highest UV absorbance values.

Table 1 summarizes the SPF (sun protection factor) value (determined as described hereinabove) of the ethanol leaf extracts of various plants.

TABLE 1
Sun protection factor (SPF) values for ethanol
extracts (0.0833 mg/ml) of leaves of various plants
from the Judean Desert (mean ± SEM, n = 3)

| Species | SPF |
|---|---|
| Achillea fragantissima | 0.50 ± 0.07 |
| Salix acmophylla | 0.54 ± 0.05 |
| Cupressus sempervirens | 0.44 ± 0.03 |
| Pistacia lentiscus | 2.17 ± 0.03 |
| Vitis vinifera | 0.63 ± 0.01 |
| Myrtus communis | 1.71 ± 0.05 |
| Salvia officinalis | 0.70 ± 0.02 |
| Thymelaea hirsuta | 0.52 ± 0.04 |
| Olea europaea | 0.54 ± 0.08 |
| Pinus pinea | 0.21 ± 0.01 |
| Rosmarinus officinalis | 1.20 ± 0.18 |
| Artemisia sp. | 0.26 ± 0.01 |
| Rhus coriaria | 7.51 ± 0.45 |
| Dittrichia viscosa | 1.37 ± 0.12 |
| Ficus carica | 0.26 ± 0.04 |
| Chiliadenus iphionoides | 1.55 ± 0.14 |
| Helichrysum italicum | 2.00 ± 0.24 |
| Verbascum sinaiticum | 0.05 ± 0.01 |
| Withania somnifera | 0.64 ± 0.03 |
| Origanum syriacum | 0.82 ± 0.02 |

As shown in Table 1, the ethanol extract of tanner's sumac (Rhus coriaria) exhibited by far the highest SPF values in vitro (protection from UVB radiation) from among the tested plants.

Extracts of tanner's sumac were therefore subjected to further testing. Extracts of different parts of the sumac were prepared from sumac from various cultivating areas.

As shown in FIG. 1, extracts of the leaves and budding fruit of sumac were considerably most effective at filtering UV light than were extracts of sumac branches, twigs, ripe fruits or roots.

### Example 2

### Isolation of UV-Blocking Substance from Sumac Leaves

From among the various sumac leaf extracts described in Example 1 hereinabove, the most effective extract (from a specimen from the Kiryat Arba region) was further purified by subjecting it to several isolation techniques. After each step, the obtained fraction was evaluated based on its UV absorbance value, in order to isolate a UV-absorbing component, referred to herein as “SH-101”. Its purity was determined by HPLC, according to procedures described hereinabove.

Following laborious experimentation, the following general extraction procedure was designed in order to maximize yield efficiency.

As shown in FIG. 2, extraction 100 comprises, in a first step, obtaining a dried powder 10 of Rhus coriaria leaves, e.g., by grinding. The dried powder may optionally be obtained according to any suitable technique known in the art. Following removal of water and volatile compounds in Rhus coriaria leaves, the weight of dried powder 10 may be about 35±5 weight percents of the weight of the undried leaves.

In a next step, dried powder 10 is contacted with ethanol, optionally warm ethanol (e.g., using a Soxhlet extractor), and the ethanol is then evaporated, to obtain an ethanolic extract 20 of Rhus coriaria leaves.

The (weight-to-volume) ratio of dried powder 10 to ethanol is optionally about 1:20 (1 gram of dried powder 10 to 20 ml ethanol). At this ratio, a yield of 39.64±2.35% (weight of ethanolic extract 20 relative to weight of dried powder 10) was obtained.

As shown in FIG. 3, upon extraction of 50 grams dry sumac leaf powder with various volumes of ethanol, the use of more than about 1000 ml ethanol was associated with lower SPF value and higher yield (weight of extracted material), whereas the use of less than about 1000 ml (and especially less than about 400 ml) was associated with similar SPF value and lower yield.

These results indicate that the use of more than about 20 ml ethanol per gram dried powder 10 resulted in extraction of significant amounts of materials which did not contribute to UV-protection, whereas the use of less than about 20 ml (and especially less than about 8 ml) ethanol per gram dried powder 10 resulted in extraction of significantly less UV-absorbing material.

In a next step, ethanolic extract 20 is subjected to the steps of being suspended in distilled water 22, partitioned with n-hexane/water 24, and the water extract obtained from step 24 is partitioned with ethyl acetate 26, so as to obtain ethyl acetate extract 30. Upon partitioning with n-hexane/water 24, a green-black solid may be extracted into the hexane phase.

Suspension in distilled water 22 and partitioning 24 are optionally effected using proportions of about 1 gram ethanolic extract 20 to about 20 ml water to about 5 ml hexane (e.g., about 50 grams ethanolic extract 20, about 1000 ml water, and about 250 ml hexane). Partitioning 26 is optionally effected using proportions of 1 ml water to 1.5 ml ethyl acetate (e.g., about 1000 ml water, and about 1500 ml ethyl acetate). Using these proportions, a yield of 26.46±3.78% (weight of ethyl acetate extract 30 relative to weight of ethanolic extract 20) was obtained.

Ethyl acetate extract 30 is then subjected to crystallization 32 in a mixture of ethanol and water, optionally at a ratio of 40:60 (ethanol:water), followed by filtration and solvent evaporation 34, to obtain a pooled dried crystallization supernatant 40.

Crystallization 32 is optionally effected at a ratio of extract 30 to solvent (ethanol/water) of about 0.1 mg extract 30 per ml solvent. Using this ratio, a maximized yield of 34.8±4.2% (weight of pooled dried crystallization supernatant 40 relative to weight of ethyl acetate extract 30) was obtained.

Pooled dried crystallization supernatant 40 is optionally subjected to reverse phase column chromatography 42, optionally using a C18 column eluted with an aqueous solution of 0.1% trifluoroacetic acid (TFA) in an acetonitrile gradient from 4% to 25% acetonitrile, to thereby obtain pooled pure SH-401 50. A chromatography yield of 30.15±2.34% (weight of SH-401 50 relative to weight of pooled dried crystallization supernatant 40) was obtained.

In exemplary extractions using the above general procedures, an overall SH-401 yield of 0.452±0.082% was obtained (weight of SH-401 relative to weight of undried sumac leaves). This simple, but elegant procedure results in high yields of the compound, at an overall cost which is comparable to chemically related commercially available compounds. Additional increases in yields may be obtained, for example, by optimization of the plant's environmental agricultural parameters (e.g., using a strain characterized by high production of SH-101), thereby further reducing the overall cost of the material.

### Example 3

### Characterization of UV-Blocking Substance (SH-401) from Sumac Leaves

SH-101 was isolated from sumac leaves, using procedures such as described in Example 2 hereinabove. The purified SH-101 was then characterized by HPLC and various spectroscopic techniques.

As shown in FIGS. 4A and 4B, a fraction with a highly pure product was obtained (retention time=107.9 minutes), as determined by HPLC analysis (99.79% purity by peak area %).

As shown in FIG. 4C, the obtained fraction exhibited strong absorption in the relevant UVB region (290-320 nm), with absorption peaks at 217 and 279.5 nm.

The isolated compound was obtained as a white, largely amorphous powder, having a melting point in a range of 220−250° C.

The SH-401 exhibited an intense blue color in the presence of FeCl₃ and a reddish color in the presence of KIO₃. These color reactions are similar to those reported for gallotannins [Haddock et al., J Chem Soc Perkin Trans 1 1982, 0:2535-2545].

In addition, the infrared (KBr) spectrum (not shown) exhibited absorption at 3950 cm⁻¹ and 1712 cm⁻¹, which are consistent with hydroxyl and carbonyl groups, respectively, in galloyl moieties.

Taken together, the above color reactions and UV and infrared spectra indicate that SH-401 is a gallotannin.

The purified fraction was further analyzed using by mass spectrometry, utilizing both liquid chromatography-mass spectrometry (LC-MS) and high-resolution mass spectrometry, using a Q-TOF 6545 (High Resolution) LC-MS (ESI/APCI/ASAP) mass spectrometer (Agilent).

As shown in Table 2 below, the mass spectrum obtained by LC-MS included peaks at about 939 Da and about 469 Da, corresponding to (M-H)⁻ and (M-2H)⁻² ions, respectively, and suggesting that SH-401 has the formula C₄₁H₃₂O₂₆.

TABLE 2
Liquid chromatography-mass spectrometry results for SH-101

| | Cal. | Diff. | | | | |
|---|---|---|---|---|---|---|
| m/z | m/z | (ppm) | z | Abundance | Formula | Ion |
| 469.05147 | 469.05181 | 0.74 | 2 | 15273.89 | C₄₁H₃₂O₂₆ | (M − 2H)⁻² |
| 469.55345 | 469.55351 | 0.12 | 2 | 8992.33 | C₄₁H₃₂O₂₆ | (M − 2H)⁻² |
| 470.05553 | 470.05477 | −1.62 | 2 | 4023.56 | C₄₁H₃₂O₂₆ | (M − 2H)⁻² |
| 470.55781 | 470.55612 | −3.6 | 2 | 1333.78 | C₄₁H₃₂O₂₆ | (M − 2H)⁻² |
| 939.10911 | 939.1109 | 1.91 | 1 | 1585.57 | C₄₁H₃₂O₂₆ | (M − H)⁻ |
| 940.11291 | 940.1143 | 1.48 | 1 | 985.82 | C₄₁H₃₂O₂₆ | (M − H)⁻ |
| 941.11738 | 941.11682 | −0.59 | 1 | 429.47 | C₄₁H₃₂O₂₆ | (M − H)⁻ |
| 942.12125 | 942.11951 | −1.85 | 1 | 149.48 | C₄₁H₃₂O₂₆ | (M − H)⁻ |

Importantly, the molecular weight of SH-101 is more than three times that of conventional commercial UV absorbers, a factor that is likely to reduce penetration potential into skin and the potential systemic deleterious effects.

Furthermore, the mass spectrum obtained by Q-TOF mass spectrometry included an ion peak at m/z 939.09 (consistent with the above LC-MS results), and further included an ion peak at m/z 168.46, which corresponds to the mass of a gallate anion (169 Da); an ion peak at m/z 769.08, which corresponds to the expected mass resulting from neutral loss of gallic acid (loss of 170 Da); and a set of ion peaks from m/z 331.07 to m/z 939.09 which were separated by a constant difference of 152, corresponding to the expected mass of a galloyl moiety (C₇H₄O₄).

The above results are consistent with the description of Berardini et al. [Rapid Commun Mass Spectrom 2004, 18:2208-2216] of fragmentation pathways associated with gallotannins (in mango peels, pulp and kernels) involving neutral loss of gallic acid (loss of 170.02 Da) and galloyl fission (loss of 152.01 Da).

Thus, the above results indicate that SH-401 comprises multiple galloyl moieties, and has a molecular weight of about 940 Da; and suggest a compound comprising five galloyl groups and an additional moiety with a molecular weight of 180 Da (e.g., a hexose, wherein SH-101 is C₄₁H₃₂O₂₆).

The structure of the SH-401 gallotannin was determined by ¹H-NMR and ¹³C-NMR 1D spectral data (obtained at 700 MHz, in CD₃OD) as well as by DEPT, COSY, HMBC and HMQC 2D-NMR.

As detailed below, the SH-401 was determined based on NMR data analysis to have the structure of 1,2,3,4,6-pentagalloyl-β-D-glucose (β-D-glucopyranose, pentakis(3,4,5-trihydroxybenzoate), as depicted in FIG. 5. The assignment of ¹H and ¹³C chemical shifts is summarized in Table 3 below. The aforementioned structure is also supported by the UV absorption, infra-red and mass spectroscopy data discussed hereinabove.

The ¹H-NMR spectrum included five aromatic singlets, in the spectral region between 6.5 and 7.15 ppm, which were consonant with the presence of five aromatic moieties assigned to the five magnetically non-equivalent protons of the galloyl groups in the molecule.

A second set of protons, represented by resonances appearing between 4.35 and 6.25 ppm, was attributed to the seven carbon-attached protons of a glucopyranosyl moiety. In the sugar region, the spectrum showed five clearly downfield-shifted proton resonances. One ¹H signal, a doublet at 6.23 ppm with a large coupling constant, could be attributed to an α,β-configured glucose anomeric proton. A ¹H triplet at 5.90 ppm and three ¹H signals at 5.61 ppm (d, J=12 Hz) and 4.40 ppm (dd, J=12, 44 Hz) and 5.58 ppm were assigned to H-2 and H-4 and H-6 glucose protons. The signals of these protons are significantly downfield compared with those in β-D-glucopyranose, suggesting the location of galloyl units at these centers.

The 2H signals of the glucose methylene group appeared at 4.51 and 4.37 as shown by HMBC experiment. DEPT NMR data showed that SH-401 contains precisely one methylene (CH₂) group.

TABLE 3
Correlation of chemical shifts observed by ¹H-NMR and
¹³C-NMR to atoms of 1,2,3,4,6-pentagalloyl glucose.

| | δ_{H} | δ_{C} |
|---|---|---|
| Glucose | | |
| 1 | 6.23 | 93.89 |
| 2 | 5.58 | 72.26 |
| 3 | 5.61 | 69.88 |
| 4 | 5.89 | 74.18 |
| 5 | 4.40 | 74.50 |
| 6 | 4.51, 4.37 | 63.19 |
| Gallate ester-1 | | |
| C═O | — | 166.27 |
| i | — | 119.81 |
| o | 7.05 | 110.69 |
| m | — | 146.61 |
| p | — | 140.81 |
| Gallate ester-2 | | |
| C═O | — | 167.08 |
| i | — | 120.32 |
| o | 6.95 | 110.48 |
| m | — | 146.43 |
| p | — | 140.36 |
| Gallate ester-3 | | |
| C═O | — | 166.99 |
| i | — | 120.28 |
| o | 6.98 | 110.53 |
| m | — | 146.50 |
| p | — | 140.41 |
| | — | — |
| Gallate ester-4 | | |
| C═O | — | 167.36 |
| i | — | 120.44 |
| o | 6.90 | 110.45 |
| m | — | 146.34 |
| p | — | 140.18 |
| Gallate ester-6 | | |
| C═O | — | 167.99 |
| i | — | 121.12 |
| o | 7.11 | 110.40 |
| m | — | 146.53 |
| p | — | 140.06 |

A ¹H-¹H homonuclear COSY experiment allowed the identification of the coupling network of the glucose moiety from H-1 to H-6a/b, as shown in Table 3. In addition, an observed correlation between the proton at 4.37 ppm and the proton at 4.51 ppm confirmed the assignment of these protons to the glucose methylene (CH₂) group.

The ¹³C-NMR spectrum exhibited signals (6c) at 93.89, 72.26, 69.88, 74.18, 74.50 (5 anomeric C); 63.19 (glucose methylene C); 166.27 (gallate ester 1, C═O); 119.81, 110.69, 146.61, 146.61 (gallate ester 1, C); 167.08 (gallate ester 2, C═O); 120.32, 110.48, 146.43, 140.36, (gallate ester 2, C); 166.99 (gallate ester 3, C═O); 120.28; 110.53; 146.50; 140.41 (gallate ester 3, C); 167.36 (gallate ester 4, C═O); 120.44; 110.45; 146.34; 140.18; (gallate ester 4, C); 167.99; (gallate ester 6, C═O); 121.12; 110.40; 146.53; 140.06; (gallate ester 6, C).

An HMQC experiment for detecting one bond ¹H×¹³C correlations showed five carbon atoms (attributed to the ortho position of galloyl moieties) attached to hydrogens at chemical shifts of about 7.11, 7.05, 6.98, 6.95 and 6.90 ppm.

An HMBC experiment for detecting long-range ¹H×¹³C correlations showed that the proton with a chemical shift of about 7.05 ppm correlated with carbon atoms at about 140, 122, and 110 ppm.

Taken together, these results indicate that the hydroxyl groups at C-1, C-2, C-3, C-4 and C-6 are galloylated [De Bruyn et al., Bull Soc Chim Belges 1977, 86:259-265].

The abovementioned molecular weight is more than three times that of known commercial UV filters. This factor is likely to reduce penetration potential into skin and the associated deleterious effects.

As shown in FIG. 6, the purification procedure increased the SPF values of the obtained SH-401 by more than 10-fold at 0.01 mg/ml relative to that of the crude extract, from 0.67±0.09 for the crude Rhus coriaria mixture to 9.09±0.72 for the purified SH-401 fraction.

This result indicates the superiority of the isolated agent over the crude extract mixture as a sunscreen agent, thus confirming the importance of SH-401 as a UV-absorbing component of sumac.

As further shown in FIG. 6, the SPF value of SH-401 (at 0.01 mg/ml) was at least as high as that of all commercially available synthetic UV filters tested.

In order to evaluate photostability, uniform films of pure SH-401 were prepared on PMMA (polymethyl methacrylate) slides by depositing 2 ml of SH-101 solution on the slides and evaporating the solvent. The samples were then exposed to UV radiation for various time intervals, ranging up to 25 hours. The glass plates were then immersed in a mixture of ethanol/water (2:98% v/v) and the films were dissolved ultrasonically. The UV samples were then quantified by UV-visible absorption spectroscopy and the SPF value was determined according to procedures described hereinabove and plotted as a function of time.

As shown in FIG. 7, the SH-401 exhibit considerably stability towards UV radiation, with essentially no change in SPF value upon 12 hours of exposure to UV radiation.

Taken together, the above results indicate that SH-401 exhibits sufficient UV absorption and photostability to serve as an effective sunscreen agent.

### Example 4

### Effect of SH-401 from Sumac Leaves on Human Skin Cells

In order to investigate the safety and efficacy of the active compound SH-401 (isolated as described in Example 2), several experiments were performed on human skin explants, according to procedures described in the Materials and Methods section hereinabove.

As shown in FIG. 8A, topical application of the SH-401 did not compromise skin viability, as determined by MTT assay, over the entire concentration range tested (up to maximal solubility). This result indicates that SH-401 is well-tolerated by human skin.

As shown in FIGS. 8B and 8C, SH-401 attenuated UVB-induced apoptosis in the epidermis (FIG. 8B) and TNFα hypersecretion (FIG. 8C), as determined by a caspase-3 activation assay and ELISA assay, respectively. As further shown therein, the effect of the SH-401 was comparable to that of a commercially available formulation, Ultrasol™ SPF 30 formulation (Dr. Fisher), used as a control.

A common feature of UVB-induced damage is the generation of ROS (reactive oxygen species), which increases DNA damage, amplifies the generation of lipid peroxidation and hampers proteins and membranes within the cells [Schuch et al., Free Rad Biol Med 2017, 107:110-124]. The SH-401 was therefore tested for its effect on UVB-induced ROS generation in skin cells, which was hypothesized to be a factor in counteracting the deleterious impact of UVB radiation. ROS generation upon UVB radiation was evaluated in skin explants both by DCFDA (dichlorofluorescin diacetate) assay, as well as by measuring lipid peroxidation (by ELISA), which is associated with ROS generation.

As shown in in FIG. 9, SH-401 attenuated UVB-induced ROS generation in skin cells in a dose-dependent manner, as determined by DCFDA assay.

In addition, as shown in FIG. 10, SH-401 attenuated UVB-induced lipid peroxidation in skin cells in a dose-dependent manner.

The above results indicate that SH-401 attenuates ROS formation in skin cells by UVB radiation.

Antioxidant capacity was further evaluated using the DPPH (diphenylpicrylhydrazyl) method. The color shift of DPPH in the presence of SH-401 and converted to units of Trolox (6-hydroxy-2,5,7,8-tetramethylchroman-2-carboxylic acid) equivalent antioxidant capacity based on a calibration curve.

As shown in FIG. 11, SH-401 is a strong antioxidant, with a 1 weight percent solution exhibiting approximately 2,310 micromole Trolox equivalent (TE) units per 100 grams.

These results indicate that the protection from UV radiation exhibited by SH-401 is mediated, at least in part, by antioxidant properties of the compound.

In addition, DNA fragmentation of similarly treated samples was determined by the COMET (single cell gel electrophoresis) assay, using an OxiSelect™ Comet Assay Kit according to the manufacturer's instructions.

As shown in FIG. 12, SH-401 attenuated UVB-induced DNA damage, as determined by COMET assay.

As shown in FIG. 13, SH-401 reduced UVB-induced formation of cyclobutane pyrimidine dimers (CPD), the primary type of DNA mutagenesis caused by UVB.

The effect of SH-401 on cells was further assessed by histological examination of treated skin explants.

As shown in FIGS. 14A-14D, UVB radiation induced formation of pyknotic “sunburn cells” and reduction of the epidermal layer (FIG. 14B), and SH-401 reversed this deleterious effect of the radiation.

These results indicate that SH-401 is effective at protecting human skin from UV radiation.

### Example 5

### Effect of SH-401 on Skin Aging and Wound Healing

The effect of SH-401 on skin aging was assessed, by examining two important parameters in the extracellular matrix balance, which are affected in skin aging and wrinkle formation: collagen synthesis following environmental insult; and activity of matrix metalloproteinase-1 (MMP1), a key enzyme in collagen degradation.

As shown in FIGS. 15A and 15B, SH-401 enhanced collagen synthesis and reduced MMP1 activity in a dose-dependent manner, following environmental insult (UVB radiation).

These results indicate that SH-401 exhibits anti-aging properties in skin.

The effect of SH-401 on wound healing in skin was assessed using a confluent HaCaT cell in vitro model of wound closure.

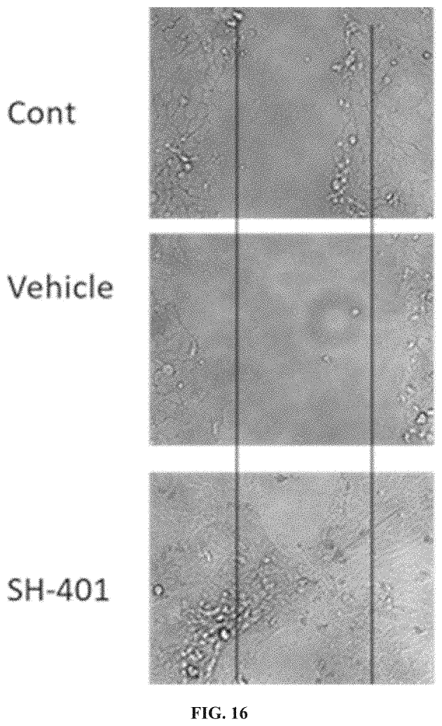

As shown in FIG. 16, SH-401 enhanced wound closure in an in vitro model.

These results indicate that SH-401 promotes wound healing.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A sunscreen composition comprising a UV-absorbing material and a dermatologically acceptable carrier, wherein said UV-absorbing material is extracted from sumac (Rhus spp.) leaves and/or budding fruits, and a concentration of said UV-absorbing material in the composition is at least 0.005 mg/ml.

2. The composition of claim 1, wherein a concentration of said UV-absorbing material in the composition is sufficient to absorb at least 90% of radiation at a wavelength of 300 nm over a path length of 1 mm.

3. (canceled)

4. The composition of claim 1, wherein said UV-absorbing material comprises at least one compound having a glucose moiety with a plurality of galloyl substituents.

5. The composition of claim 4, wherein said at least one compound comprises 1,2,3,4,6-pentagalloyl glucose.

6. The composition of claim 1, wherein said UV-absorbing material is obtainable by a process comprising contacting said sumac leaves and/or budding fruits with a water-miscible organic solvent, and removing said water-miscible organic solvent, to thereby obtain said UV-absorbing material.

7.-21. (canceled)

22. A pharmaceutical or cosmetic composition comprising a UV-absorbing material extracted from sumac (Rhus spp.) leaves and/or budding fruits, and a dermatologically acceptable carrier.

23. (canceled)

24. The composition of claim 22, comprising at least one compound having a glucose moiety with a plurality of galloyl substituents.

25. (canceled)

26. A method of treating a condition selected from the group consisting of skin aging and a wound in a subject in need thereof, the method comprising administering to the subject the composition of claim 22.

27. The composition of claim 22, wherein said cosmetic composition is a skin rejuvenation composition and/or a peeling composition.

28. A process for obtaining a UV-absorbing material, the process comprising contacting sumac (Rhus spp.) leaves and/or budding fruits with a water-miscible organic solvent, and removing said water-miscible organic solvent, to thereby obtain said UV-absorbing material.

29. The process of claim 28, wherein said water-miscible organic solvent comprises a C₁₋₄-alcohol.

30. (canceled)

31. The process of claim 28, wherein said contacting comprises extraction effected using a Soxhlet extractor.

32. The process of claim 28, wherein said contacting is effected using a ratio of from 5 to 40 ml of said water-miscible organic solvent per gram dry weight of said sumac leaves and/or budding fruits.

33. The process of claim 28, further comprising partitioning said UV-absorbing material in a polar solvent and a nonpolar solvent, and collecting the fraction which partitions into said polar solvent.

34. The process of claim 33, wherein said nonpolar solvent comprises an alkane and/or said polar solvent comprises water.

35.-36. (canceled)

37. The process of claim 33, further comprising partitioning said fraction which partitions into said polar solvent in water and a water-immiscible polar organic solvent, and collecting the fraction which partitions into said water-immiscible polar organic solvent.

38.-42. (canceled)

43. The process of claim 28, wherein said UV-absorbing material comprises at least one compound having a glucose moiety with a plurality of galloyl substituents.

44. The process of claim 43, wherein said at least one compound comprises 1,2,3,4,6-pentagalloyl glucose.

45. The process of claim 44, wherein a purity of said 1,2,3,4,6-pentagalloyl glucose in said UV-absorbing material is at least 95 weight percents.

46. A UV-absorbing material obtained according to the process of claim 28.
